(19) United States
(12) Patent Application Publication
(10) Pub. No.: US 2022/0064587 A1
(43) Pub. Date: Mar. 3, 2022

(54) MICROALGAE-CONTAINING PRODUCT AND PRODUCTION METHOD THEREFOR

(71) Applicant: OP Bio Factory Co., Ltd., Okinawa (JP)

(72) Inventors: Akihiko KANAMOTO, Okinawa (JP); Takeshi FUJIWARA, Okinawa (JP); Takafumi WATANABE, Okinawa (JP)

(21) Appl. No.: 17/418,611

(22) PCT Filed: Nov. 18, 2019
(86) PCT No.: PCT/JP2019/045103

§ 371 Date: Jun. 25, 2021

(30) Foreign Application Priority Data
Dec. 28, 2018 (JP) 2018-248155
Aug. 6, 2019 (JP) 2019-144715

(51) Int. Cl. C12N 1/12; A61K 8/9706; A61Q 5/00; A61Q 19/00; A23L 17/60; A23G 9/36; A23G 9/44

(52) U.S. Cl. CPC C12N 1/12; A61K 8/9706; A61Q 5/00; A23V 2002/00; A23L 17/60; A23G 9/363; A23G 9/44; A61Q 19/00

(57) ABSTRACT

The present invention provides a microalgae product and a production method for the microalgae product. In one embodiment, the present invention provides a method for treating microalgae without an increase in pheophorbide. By this method, provided is a safe microalgae product having reduced pheophorbide. Such a microalgae product provides various healthy, nutritional, and/or cosmetic effects. The present invention also provides a culturing device that enables high-concentration culturing with less bacterial contamination, and thereby enables highly useful culturing of microalgae.

### TECHNICAL FIELD

The present disclosure relates to a product (e.g., an edible product or a cosmetic product) containing microalgae, and a method and system for the manufacture thereof. The present disclosure particularly relates to a product (e.g., an edible product or a cosmetic product) containing microalgae with reduced pheophorbide, a method of recovering/concentrating microalgae that enables the provision thereof, and an apparatus for culturing microalgae at a high concentration.

### BACKGROUND ART

Microalgae such as Chlorella and Euglena have drawn attention for nutritional components contained therein such as vitamins and minerals, and are utilized as health food or ingredients of food products. For Chlorella, which is currently widely distributed as food products, various culturing methods have been established, such as an autotrophic culturing method through photosynthesis and heterotrophic culturing method utilizing an organic carbon source. However, what needs to be heeded upon handling such as the growth rate, size, and presence/absence of cell walls vary depending on the type of microalgae. Therefore, there is a demand for establishing manufacturing methods matching the properties of cells.

Microalgae can contain various components including useful components and harmful components. To provide safe food products, harmful components may need to be reduced. While contained components can be adjusted by selective breeding of microalgae, such as Chlorella with high chlorophyll content (Patent Literature 1=Japanese Laid-Open Publication No. 2016-67313), the components contained in a product can also vary due to differences in the manufacturing method. It is desirable that a manufacturing method is established which would result in high useful component content and low harmful component content, and products (e.g., food products) produced by such a method are provided.

### CITATION LIST

### Patent Literature

[PTL 1] Japanese Laid-Open Publication No. 2016-67313

### SUMMARY OF INVENTION

### Solution to Problem

As a result of diligent studies, the inventors found a method of efficiently manufacturing a high quality microalgal product. The present disclosure can also provide a microalgal product (e.g., edible product or cosmetic product) that is safe and has a new functionality. The manufacturing method of the present disclosure can provide a microalgal product with reduced pheophorbide by applying treatment for inactivating and/or decomposing an enzyme that has an adverse effect on microalgae themselves or a microalgal component to microalgae with reduced disruption in the cell membrane and/or cell wall such as a rupture, or reduced secretion and/or activity of an enzyme that promotes disruption of an organelle, autodigestion, intracellular component breakdown, or the like (e.g., by reducing physical damage and/or chemical damage).

The present disclosure provides a method of efficiently manufacturing a high quality microalgal product and an apparatus that can be advantageously used in such a method.

Therefore, the present disclosure typically provides the following.

### (Item B1)

A method for manufacturing a microalgal product, comprising:
(A) a step of maintaining microalgae under a condition where an amount of stress applied to the microalgae from after culture to step (B) is controlled to a predetermined value or less, wherein the step maintains a density of the microalgae at or below a predetermined value and/or does not concentrate the microalgae by a predetermined factor or greater; and
(B) a step of subjecting the microalgae to treatment for inactivating a chlorophyllase.

### (Item B2)

The method of any of the preceding items, wherein the predetermined value of the density and/or the predetermined factor of the concentration is determined based on an increase in pheophorbide upon concentrating the microalgae.

### (Item B3)

The method of any of the preceding items, wherein the predetermined value of the density is less than or equal to about 10 g/L (dry weight).

### (Item B4)

The method of any of the preceding items, wherein the predetermined value of the density is less than or equal to about 5 g/L (dry weight).

### (Item B5)

The method of any of the preceding items, wherein the predetermined factor of the concentration is greater than or equal to about 100-fold.

### (Item B6)

The method of any of the preceding items, wherein the predetermined factor of the concentration is greater than or equal to about 10-fold.

### (Item B7)

The method of any of the preceding items, wherein the predetermined value of the amount of stress is less than or equal to about 5.

### (Item B8)

The method of any of the preceding items, wherein the predetermined value of the amount of stress is less than or equal to about 3.

### (Item B9)

The method of any of the preceding items, wherein the predetermined value of the amount of stress is less than or equal to about 2.

### (Item B10)

The method of any of the preceding items, wherein treatment for concentrating the microalgae is not performed in step (A).

### (Item B11)

The method of any of the preceding items, wherein a step of culturing the microalgae comprises growing the microalgae to a density greater than or equal to 1 g/L (dry weight).

### (Item B12)

The method of any of the preceding items, comprising a step of concentrating the microalgae after step (B).

### (Item B13)

The method of any of the preceding items, wherein step (B) comprises heating the microalgae.

### (Item B14)

The method of any of the preceding items, wherein the heating comprises heating to 95° C. or higher.

### (Item B15)

The method of any of the preceding items, wherein step (B) is performed under a condition that does not decompose fucoxanthin, or where a reduction in fucoxanthin after step (B) in comparison to before the step is less than 80%.

### (Item B16)

The method of any of the preceding items, wherein the condition comprises an amount of decomposition of fucoxanthin of less than 10%.

### (Item B17)

The method of any of the preceding items, comprising a step of drying the microalgae after step (B).

### (Item B18)

The method of any of the preceding items, wherein the microalgae produce 30 mg or more chlorophyll per 1 g of dry weight.

### (Item B19)

The method of any of the preceding items, wherein the microalgae are algae that produce fucoxanthin.

### (Item B20)

The method of any one of the preceding items, wherein the microalgae are algae that produce 8 mg or more fucoxanthin per 1 g of dry weight.

### (Item B21)

The method of any of the preceding items, wherein the microalgae are microalgae of the class Haptophyceae.

### (Item B22)

The method of any of the preceding items, wherein the microalgae are microalgae of the family Pavlovaceae.

### (Item B23)

The method of any of the preceding items, wherein the microalgae are microalgae of the genus Pavlova.

### (Item B24)

The method of any of the preceding items, wherein the microalgae are P. calceolate, P. granifera, P. gyrans, P. lutheri, P. pinguis, or P. salina.

### (Item B25)

The method of any of the preceding items, wherein the microalgae are P. granifera or P. gyrans.

### (Item B26)

A microalgal product comprising an algal body of the microalgae for use in an organism or for intake by an organism, manufactured by a method comprising performing the method of any of the preceding items.

### (Item B27)

The microalgal product of any of the preceding items, wherein pheophorbide content of the microalgae is less than or equal to 0.2% by weight.

### (Item B28)

The microalgal product of any of the preceding items, wherein the pheophorbide content of the microalgae is less than or equal to 0.1% by weight.

### (Item B29)

A microalgal product for use in an organism or for intake by an organism, comprising an algal body of microalgae, wherein pheophorbide content of the microalgae is less than or equal to 0.2% by weight (dry weight).

### (Item B30)

A microalgal product for use in an organism or for intake by an organism, comprising an algal body of microalgae, wherein pheophorbide content of the microalgae is less than or equal to 0.1% by weight (dry weight).

### (Item B31)

The microalgal product of any of the preceding items, wherein the microalgae are microalgae of the class Haptophyceae.

### (Item B32)

The microalgal product of any of the preceding items, wherein the microalgae are P. granifera or P. gyrans.

### (Item B33)

The microalgal product of any of the preceding items, which is an edible product or a cosmetic product.

### (Item B34)

The microalgal product of any of the preceding items, wherein the organism is a mammal.

### (Item B35)

The microalgal product of any of the preceding items, wherein the organism is a human.

### (Item B36)

The microalgal product of any of the preceding items, wherein fucoxanthin content is greater than or equal to 0.8% by weight.

### (Item B37)

The microalgal product of any of the preceding items, wherein fucoxanthin content of the microalgae is greater than or equal to 0.8% by weight (dry weight).

### (Item B38)

The microalgal product of any of the preceding items, wherein chlorophyll content of the microalgae is greater than or equal to 3% by weight (dry weight).

### (Item B39)

The microalgal product of any of the preceding items, which is an edible product.

### (Item B40)

The microalgal product of any of the preceding items, which is a food product.

### (Item B41)

The microalgal product of any of the preceding items, which is an edible product that is taken to provide 100 to 150 mg of chlorophyll per day.

### (Item B42)

The microalgal product of any of the preceding items, which is a cosmetic product.

### (Item B43)

A method for manufacturing a frozen product, comprising:

a step of preparing a microalgal concentrate by the method of any of the preceding items; and

a step of freezing the microalgal concentrate.

### (Item B44)

The method of any of the preceding items, wherein the freezing step comprises cooling to −40° C. or lower.

### (Item B45)

The microalgal product of any of the preceding items, which is a frozen product.

### (Item B46)

The microalgal product of any of the preceding items, which is dairy product-free, a LACT-ICE, an ICE-MILK, or an ICE-CREAM.

### (Item B47)

The frozen product of any of the preceding items, comprising one or more of an excipient, an antioxidant, an emulsifier, and a thickener.

### (Item B48)

The frozen product of any of the preceding items, comprising one or more of fruit juice and flavoring.

### (Item B49)

The frozen product of any of the preceding items, which is in a sheet-like form.

### (Item B50)

A method for manufacturing an oil immersed product, comprising:

a step of preparing a microalgal concentrate by the method of any of the preceding items; and

a step of mixing the microalgae with oil.

### (Item B51)

The method of any of the preceding items, comprising a step of adding water to the microalgal concentrate and desalinating.

### (Item B52)

The method of any of the preceding items, comprising a step of lyophilizing the microalgal concentrate.

### (Item B53)

The microalgal product of any of the preceding items, which is an oil immersed product.

### (Item B54)

The oil immersed product of any of the preceding items, comprising an antioxidant.

### (Item B55)

The oil immersed product of any of the preceding items, wherein the antioxidant comprises α-tocopherol.

### (Item B56)

The oil immersed product of any of the preceding items, comprising about 1 to 100% by weight of oil with respect to 1 g of dry algal body.

### (Item B57)

The oil immersed product of any of the preceding items, comprising an emulsifier.

### (Item B58)

An edible capsule comprising the oil immersed product of any of the preceding items.

### (Item B59)

The microalgal product of any of the preceding items, which is a dried product comprising one or more of a desiccant and an antioxidant.

### (Item B60)

The microalgal product of any of the preceding items, which is a dried product enclosed in a lightproof container.

### (Item B61)

A method for manufacturing dried microalgae, comprising:

a step of preparing a microalgal concentrate by the method of any of the preceding items; and

a step of drying the microalgal concentrate in the presence of one or more of an excipient, an emulsifier, and an antioxidant.

### (Item A1)

A method for manufacturing a Pavlovales order microalgal product, comprising:
(A) a step of maintaining microalgae under a condition where an amount of stress applied to the microalgae from after culture to step (B) is controlled to a predetermined value or less, wherein the step maintains a density of the microalgae at or below a predetermined value and/or does not concentrate the microalgae by a predetermined factor or greater; and
(B) a step of subjecting the microalgae to treatment for inactivating a chlorophyllase.

### (Item A2)

The method of item A1, wherein the predetermined value of the density and/or the predetermined factor of the concentration is determined based on an increase in pheophorbide upon concentrating the microalgae.

### (Item A3)

The method of item A1 or A2, wherein the predetermined value of the density is less than or equal to about 10 g/L (dry weight).

### (Item A4)

The method of item A1 or A2, wherein the predetermined value of the density is less than or equal to about 5 g/L (dry weight).

### (Item A5)

The method of any one of items A1 to A4, wherein the predetermined factor of the concentration is greater than or equal to about 100-fold.

### (Item A6)

The method of any one of items A1 to A4, wherein the predetermined factor of the concentration is greater than or equal to about 10-fold.

### (Item A7)

The method of any one of items A1 to A6, wherein the predetermined value of the amount of stress is less than or equal to about 5.

### (Item A8)

The method of any one of items A1 to A6, wherein the predetermined value of the amount of stress is less than or equal to about 3.

### (Item A9)

The method of any one of items A1 to A6, wherein the predetermined value of the amount of stress is less than or equal to about 2.

### (Item A10)

The method of any one of items A1 to A9, wherein treatment for concentrating the microalgae is not performed in step (A).

### (Item A11)

The method of any one of items A1 to A10, wherein a step of culturing the microalgae comprises growing the microalgae to a density greater than or equal to 1.5 g/L (dry weight).

### (Item A12)

The method of any one of items A1 to A11, comprising a step of concentrating the microalgae after step (B).

### (Item A13)

The method of any one of items A1 to A12, wherein step (B) comprises heating the microalgae.

### (Item A14)

The method of item A13, wherein the heating comprises heating to 95° C. or higher.

### (Item A15)

The method of any one of items A1 to A14, wherein step (B) is performed under a condition that does not decompose fucoxanthin, or where a reduction in fucoxanthin after step (B) in comparison to before the step is less than 80%.

### (Item A16)

The method of item A15, wherein the condition comprises an amount of decomposition of fucoxanthin of less than 10%.

### (Item A17)

The method of any one of items A1 to A16, comprising a step of drying the microalgae after step (B).

### (Item A18)

The method of any one of items A1 to A17, wherein the microalgae produce 30 mg or more chlorophyll per 1 g of dry weight.

### (Item A19)

The method of any one of items A1 to A18, wherein the microalgae are algae that produce fucoxanthin.

### (Item A20)

The method of any one of items A1 to A19, wherein the microalgae are algae that produce 8 mg or more fucoxanthin per 1 g of dry weight.

### (Item A21)

The method of any one of items A1 to A20, wherein the microalgae are microalgae of the family Pavlovaceae.

### (Item A22)

The method of any one of items A1 to A21, wherein the microalgae are microalgae of the genus Pavlova.

### (Item A23)

The method of any one of items A1 to A22, wherein the microalgae are P. calceolate, P. granifera, P. gyrans, P. lutheri, P. pinguis, or P. salina.

### (Item A24)

The method of item A23, wherein the microalgae are P. granifera or P. gyrans.

### (Item A25)

A microalgal product comprising an algal body of the microalgae for use in an organism or for intake by an organism, manufactured by a method comprising performing the method of any one of items A1 to A24.

### (Item A26)

The microalgal product of item A25, wherein pheophorbide content of the microalgae is less than or equal to 0.2% by weight.

### (Item A27)

The microalgal product of item A26, wherein the pheophorbide content of the microalgae is less than or equal to 0.1% by weight.

### (Item A28)

A microalgal product for use in an organism or for intake by an organism, comprising an algal body of microalgae of the order Pavlovales, wherein pheophorbide content of the microalgae is less than or equal to 0.2% by weight (dry weight).

### (Item A29)

A microalgal product for use in an organism or for intake by an organism, comprising an algal body of microalgae of the order Pavlovales, wherein pheophorbide content of the microalgae is less than or equal to 0.1% by weight (dry weight).

### (Item A30)

The microalgal product of item A28 or A29, wherein the microalgae are P. granifera or P. gyrans.

### (Item A31)

The microalgal product of any one of items A25 to A30, which is an edible product or a cosmetic product.

### (Item A32)

The microalgal product of any one of items A25 to A31, wherein the organism is a mammal.

### (Item A33)

The microalgal product of any one of items A25 to A31, wherein the organism is a human.

### (Item A34)

The microalgal product of any one of items A25 to A33, wherein fucoxanthin content is greater than or equal to 0.8% by weight.

### (Item A35)

The microalgal product of any one of items A25 to A34, wherein fucoxanthin content of the microalgae is greater than or equal to 0.8% by weight (dry weight).

### (Item A36)

The microalgal product of any one of items A25 to A35, wherein chlorophyll content of the microalgae is greater than or equal to 3% by weight (dry weight).

### (Item A37)

The microalgal product of any one of items A25 to A36, which is an edible product.

### (Item A38)

The microalgal product of any one of items A25 to A36, which is a food product.

### (Item A39)

The microalgal product of any one of items A25 to A36 which is an edible product that is taken to provide 100 to 150 mg of chlorophyll per day.

### (Item A40)

The microalgal product of any one of items A25 to A36, which is a cosmetic product.

### (Item 1)

A microalgal product for use in an organism or for intake by an organism.

### (Item 2)

The microalgal product of item 1, wherein the microalgae are haptophytes.

### (Item 3)

The microalgal product of item 1 or 2, wherein pheophorbide content is less than or equal to 0.1% by weight.

### (Item 4)

The microalgal product of any one of items 1 to 3, which is an edible product or a cosmetic product.

### (Item 5)

The microalgal product of any one of items 1 to 4, wherein the organism is a mammal.

### (Item 6)

The microalgal product of any one of items 1 to 5, wherein pheophorbide content of the microalgae is less than or equal to 0.1% by weight (dry weight).

### (Item 7)

The microalgal product of any one of items 1 to 6, wherein the microalgae are microalgae of the order Pavlovales.

### (Item 8)

The microalgal product of any one of items 1 to 7, wherein the microalgae are microalgae of the family Pavlovaceae.

### (Item 9)

The microalgal product of any one of items 1 to 8, wherein the microalgae are microalgae of the genus Pavlova.

### (Item 10)

The microalgal product of any one of items 1 to 9, wherein the organism is a human.

### (Item 11)

The microalgal product of any one of items 1 to 10, wherein fucoxanthin content is greater than or equal to 0.8% by weight.

### (Item 12)

The microalgal product of any one of items 1 to 11, wherein fucoxanthin content of the microalgae is greater than or equal to 0.8% by weight (dry weight).

### (Item 13)

The microalgal product of any one of items 1 to 12, wherein chlorophyll content of the microalgae is greater than or equal to 3% by weight (dry weight).

### (Item 14)

The microalgal product of any one of items 1 to 13, which is a cosmetic product.

### (Item 15)

The microalgal product of any one of items 1 to 13, which is an edible product.

### (Item 16)

The microalgal product of any one of items 1 to 13, which is a food product.

### (Item 17)

The microalgal product of any one of items 1 to 16, which is an edible product that is taken to provide 100 to 150 mg of chlorophyll per day.

### (Item 18)

A method for manufacturing a microalgal product, comprising:
(A) a step of subjecting microalgae to treatment for inactivating a chlorophyllase under a condition for controlling an amount of stress.

### (Item 19)

The method of item 18, wherein the amount of stress is less than 2.

### (Item 20)

The method of item 18, wherein the amount of stress is less than 3.

### (Item 21)

The method of any one of items 18 to 20, wherein a density of the microalgae in the step of subjecting the microalgae to treatment for inactivating a chlorophyllase is less than or equal to 10 g/L (dry weight).

### (Item 22)

The method of any one of items 18 to 21, wherein an amount of stress applied to the microalgae upon start of step (A) is a low amount of stress.

### (Item 23)

The method of any one of items 18 to 22, wherein after a step of culturing the microalgae, treatment for concentrating the microalgae is not performed prior to step (A).

### (Item 24)

The method of any one of items 18 to 23, comprising, after a step of culturing the microalgae, concentrating a density of the microalgae to less than or equal to 3 g/L (dry weight) prior to step (A).

### (Item 25)

The method of any one of items 18 to 24, wherein a step of culturing the microalgae comprises growing the microalgae to a density greater than or equal to 1.7 g/L (dry weight).

### (Item 26)

The method of any one of items 18 to 25, comprising a step of concentrating the microalgae after step (A).

### (Item 27)

The method of any one of items 18 to 26, wherein step (A) comprises heating the microalgae.

### (Item 28)

The method of item 27, wherein the heating comprises heating to 95° C. or higher.

### (Item 29)

The method of any one of items 18 to 28, wherein step (A) is performed under a condition that does not decompose fucoxanthin, or where decomposition thereof is reduced.

### (Item 30)

The method of item 29, wherein the condition comprises an amount of decomposition of fucoxanthin of less than 10%.

### (Item 31)

The method of any one of items 18 to 30, comprising a step of drying the microalgae after step (A).

### (Item 32)

The method of any one of items 18 to 31, wherein the microalgae produce 30 mg or more chlorophyll per 1 g of dry weight.

### (Item 33)

The method of any one of items 18 to 32, wherein the microalgae are algae that produce fucoxanthin.

### (Item 34)

The method of any one of items 18 to 33, wherein the microalgae are algae that produce 8 mg or more fucoxanthin per 1 g of dry weight.

### (Item 35)

The method of any one of items 18 to 34, wherein the microalgae are microalgae of the order Pavlovales.

### (Item 36)

The method of any one of items 18 to 35, wherein the microalgae are microalgae of the family Pavlovaceae.

### (Item 37)

The method of any one of items 18 to 36, wherein the microalgae are microalgae of the genus Pavlova.

### (Item 38)

The method of any one of items 18 to 37, wherein the microalgae are P. calceolate, P. granifera, P. gyrans, P. lutheri, P. pinguis, or P. salina.

### (Item 39)

An apparatus for culturing microalgae, comprising:

at least two culturing sections having a wall made of a material that allows transmission of light;

an upper linking section for linking upper portions of the at least two culturing sections with one another;

a lower linking section for linking lower portions of the at least two culturing sections with one another; and

at least one bubble generation device installed in at least one, but not all of the at least two culturing sections;

wherein the at least two culturing sections, the upper linking section, and the lower linking section are configured to fluid-communicably enclose a medium, and

the apparatus is installed so that the upper linking section is further away from an installation floor than the lower linking section is.

### (Item 40)

The apparatus of item 39, wherein the at least two culturing sections have an outer diameter of about 10 mm to about 1000 mm.

### (Item 41)

The apparatus of item 39 or 40, wherein the at least two culturing sections have a length of about 10 cm to about 1000 cm.

### (Item 42)

The apparatus of any one of items 39 to 41, wherein the bubble generation device is installed at a location that is closer to the lower linking section than to the upper linking section.

### (Item 43)

The apparatus of any one of items 39 to 42, wherein the apparatus is configured so that the medium contacts outside air only through a filter and the bubble generation device.

### (Item 44)

The apparatus of any one of items 39 to 43 without a power source for agitation other than the bubble generation device.

### (Item 45)

The apparatus of any one of items 39 to 44, wherein the at least two culturing sections are separate units in a relationship wherein none includes any other culturing section.

### (Item 46)

The apparatus of any one of items 39 to 45, wherein the at least two culturing sections are configured so that light is not blocked by one another.

### (Item 47)

A system for manufacturing a microalgal product comprising:

a culture vessel; and

a treatment section for applying treatment for inactivating a chlorophyllase;

wherein a section from the culturing section to the treatment section is configured to control an amount of stress applied to microalgae.

### (Item 48)

The system of item 47 configured to generate a water flow at the section from the culturing section to the treatment section by a roller pump, a Mono pump, or a diaphragm pump.

The present disclosure is intended so that one or more of the features described above can be provided not only as the explicitly disclosed combinations, but also as other combinations thereof. Additional embodiments and advantages of the present disclosure are recognized by those skilled in the art by reading and understanding the following detailed description as needed.

### Advantageous Effects of Invention

The microalgal product of the present disclosure is safe and has new functionality, and can provide health, nutritional, and/or cosmetic benefits. A component that is harmful to animals (especially humans) is reduced or eliminated in the microalgal product of the present disclosure such that harmful effects are reduced or eliminated. Thus, the function of the microalgal product can be thoroughly exerted. The manufacturing method and system of the present disclosure can efficiently provide a microalgal product with reduced pheophorbide. The culturing apparatus of the present disclosure enables culture of various microalgae at a high concentration with little bacterial contamination.

### BRIEF DESCRIPTION OF DRAWINGS

FIG. 1 shows various photobioreactors used in Example 1, which are a 100 mm diameter acrylic photobioreactor, a 200 mm diameter acrylic photobioreactor, a 250 mm diameter acrylic photobioreactor, and a 450 mm diameter polyethylene bag, respectively.

FIG. 2 shows culture vessels for the open culture used in Example 1, which are a 500 L tank (using 200 L of medium) and a 750 L raceway, respectively.

FIG. 3 shows growth upon a culture of microalgae in each culture vessel. The vertical axis indicates the dry weight of microalgae contained per 1 L of medium, and the horizontal axis indicates the number of days of culture.

FIG. 4 shows a photobioreactor capable of high concentration culture of microalgae which is optimally designed.

FIG. 5 shows the configuration of the photobioreactor apparatus in FIG. 4.

FIG. 6 shows another embodiment of a photobioreactor capable of high concentration culture of microalgae. As shown in the figure, a plurality of photobioreactors can be linked.

FIG. 7 shows an embodiment of cooling with water so that the photobioreactor in FIG. 4 would not reach a high temperature.

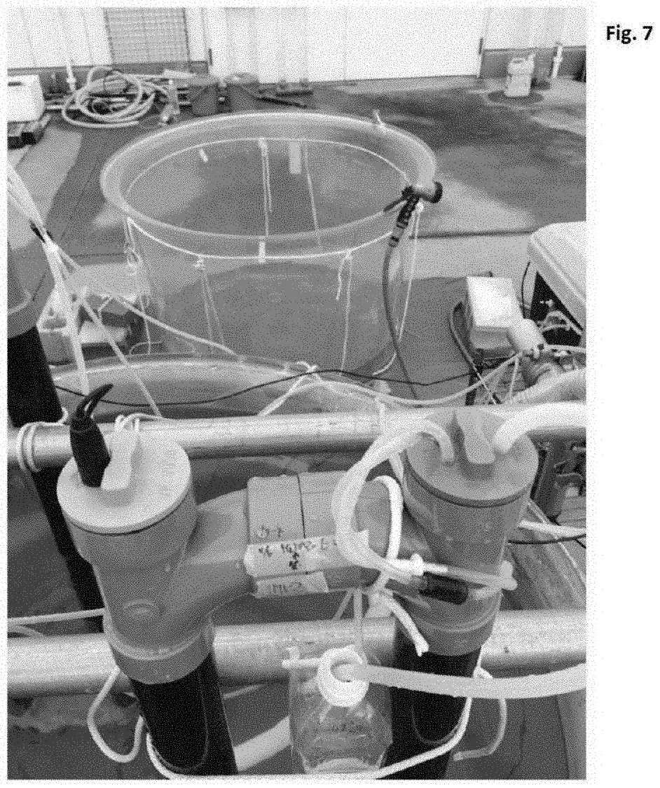

FIG. 8 shows growth upon culture of microalgae in an optimally designed photobioreactor. The vertical axis indicates the dry weight of microalgae contained in 1 L of culture, and the horizontal axis indicates the number of days of culture.

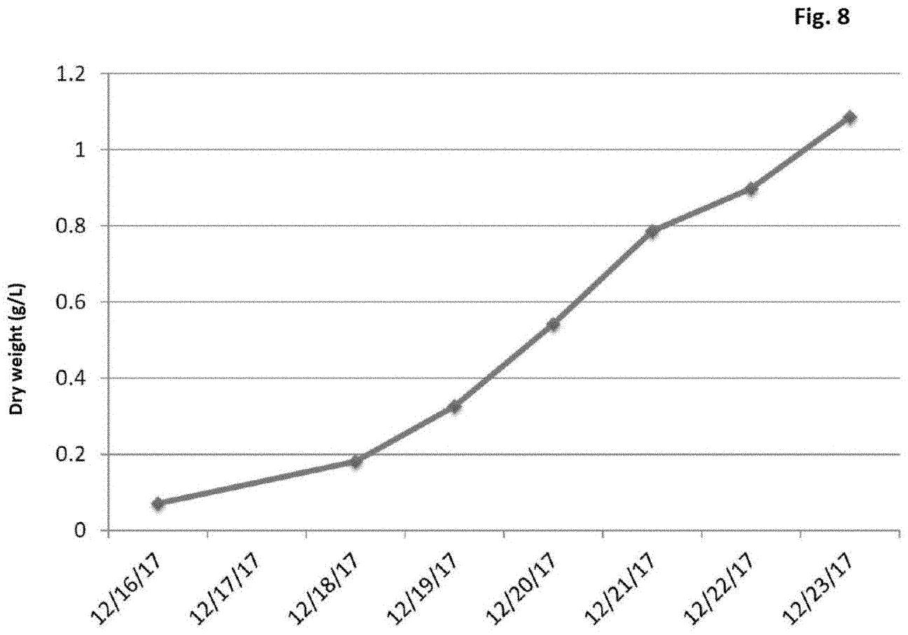

FIG. 9 shows the growth upon culture of microalgae in a photobioreactor over about 40 days. The vertical axis indicates the dry weight of microalgae contained in 1 L of culture, and the horizontal axis indicates the number of days of culture. The figure represents the culture vessels for the open culture used in Example 1

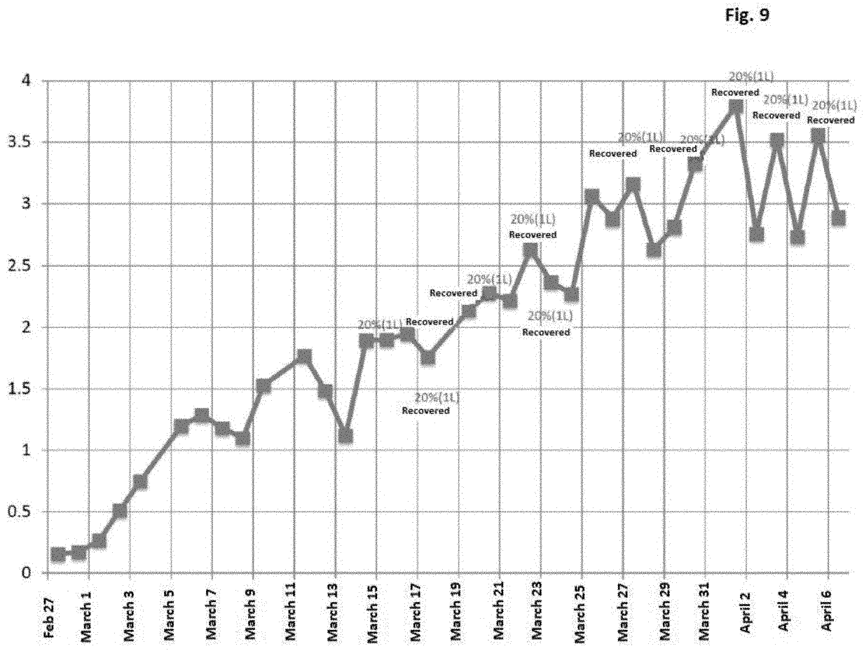

FIG. 10 shows a cascade pump used to apply a model stimulation.

FIG. 11 shows microscope images of haptophytes before and after heating. The scale bar indicates 50 μm.

FIG. 12 shows the configuration of a heating apparatus used in Example 4.

FIG. 13 shows the outer appearances of the samples before and after centrifuging the samples that underwent each of the heat treatment.

FIG. 14 shows a plate-like heating apparatus used in Example 4.

FIG. 15 shows an exemplary block diagram showing the configuration of a control unit of a system for manufacturing microalgal products by each function.

FIG. 16 shows an example of a microalgal product.

### DESCRIPTION OF EMBODIMENTS

The present disclosure is described hereinafter while showing the best mode of the present disclosure. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

The definitions of the terms and/or the detailed basic technology that are particularly used herein are described hereinafter as appropriate.

(Definitions, etc.)

As used herein, “microalgae” refers to microscopic sized (e.g., 0.1 μm to 1 mm) microorganisms comprising chloroplast that generally live in water. Microalgae include prokaryotic organisms of the phylum cyanobacteria and eukaryotic organisms of the phylum Glaucophyta, Rhodophyta (red algae), Chlorophyta, Cryptophyta (cryptophytes), Haptophyta (haptophytes), Heterokontophyta, Dinophyta (dinoflagellates), Euglenida, and Chlorarachniophyta.

The phylum Chlorophyta includes the class Trebouxiophyceae, the class Trebouxiophyceae includes the order Chlorellales, the order Chlorellales includes the family Chlorellaceae, and the family Chlorellaceae includes the genus Chlorella.

Euglenida includes the class Euglenophyceae, the class Euglenophyceae includes the order Euglenales, the order Euglenales includes the family Euglenaceae, and the family Euglenaceae includes the genus Euglena.

The phylum cyanobacteria includes the order Oscillatoriales, and the order Oscillatoriales includes the genus Arthrospira.

The phylum Haptophyta (haptophytes) includes the class Haptophyceae, and the class Haptophyceae includes the subclasses Pavlovophycidae and Prymnesiophycidae. The subclass Pavlovophycidae includes the order Pavlovales, the order Pavlovales includes the family Pavlovaceae, and the family Pavlovaceae includes Diacronema, Exanthemachrysis, Pavlova, and Rebecca. Haptophyte is a phytoplankton with a cell diameter of about 5 to 50 μm, which is an autotrophic organism performing photosynthesis. Many inhabit the ocean, but some species are also found in freshwater or brackish lake. Haptophytes constitute a large biomass in the oceanic zone and are important as a primary producer in the ocean.

As used herein, “microalgal product” refers to a product (e.g., edible product or cosmetic product) comprising the algal body of microalgae or a component of a part of microalgae. Typically, a microalgal product is a dried product, a product produced from further processing a dried product (including component extracts), or component extract manufactured from microalgae that has not been dried (e.g., fucoxanthin extract).

As used herein, “edible product” refers to an article intended for intake by an organism (e.g., animal or human). Edible products include food products and beverages used in the meaning that is commonly used, and feed for non-human animals, as well as food additives, functional food products (e.g., Food for Specified Health Uses), and supplements.

As used herein, “cosmetic product” refers to any product intended to be used by wearing, applying to the body, spraying, or other similar methods in order to clean, beautify, increase the attractiveness, or change the appearance of the body of an animal (e.g., human) or maintain young skin and healthy hair. As used herein, “cosmetic product” is not limited to “cosmetic product” specified under the so-called Act on Securing Quality, Efficacy and Safety of Products Including Pharmaceuticals and Medical Devices (former Pharmaceutical Affairs Law). A cosmetic product can also be any of, for example, a quasi-drug, a drug, or a general good. As used herein, “quasi-drug” includes products in a classification between a drug and a cosmetic product specified under the “Act on Securing Quality, Efficacy and Safety of Products Including Pharmaceuticals and Medical Devices”, which have a moderate effect on the human body, as well as mechanical instruments with a moderate effect on the human body. Examples of quasi-drugs include, but are not limited to, medicinal cosmetic products (including medicinal soap, medicinal toothpaste, and the like), bath additives, quasi-drugs for pest control (pesticides, etc.), and designated quasi-drugs (drinks, gargle, and some gastrointestinal agents, etc.). As used herein, “drugs” refer to a pharmaceutical product administered to diagnose/treat/prevent a disease in a human or animal, including those that are specified in the Japanese Pharmacopoeia, those that are intended for use in diagnosing, treating, or preventing a disease in humans or animals, which are not mechanical instruments, dental materials, medical products, or sanitation products (excluding quasi-drugs), and those intended to affect the structure or function of the body of a human or animal, which are not mechanical instruments, dental materials, medical products, or sanitation products (excluding quasi-drugs and cosmetic products).

As used herein, “chlorophyll” is used in the meaning that is commonly used in the art, which is a substance that is often used to absorb light energy in a light reaction of photosynthesis. Microalgae having chloroplasts can comprise chlorophyll.

As used herein, “pheophorbide” is used in the meaning that is commonly used in the art, which is a substance produced often by decomposition of chlorophyll in microalgae. Pheophorbide can result from a chlorophyllase acting on a chlorophyll. Due to the potential sanitary risks of inducing a dermatological disorder, the content thereof is restricted in Chlorella processed products and the like (May 8, 1981) (Env/Food No. 99) (Notice from the Director of the Environmental Health Bureau of the Ministry of Health and Welfare to prefectural governors, designated city mayors, and heads of special wards).

As used herein, “fucoxanthin” is used in the meaning that is commonly used in the art, which is a substance having the following structure:

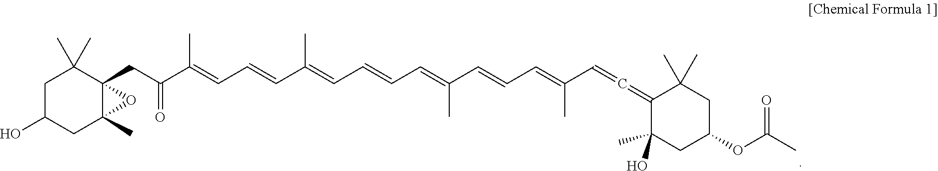 [SMILES: CC(=O)O[C@H]1CC(C)(C)C(=C=C/C(C)=C/C=C/C(C)=C/C=C/C=C(C)/C=C/C=C(\C)C(=O)C[C@@]23O[C@]2(C)CC(O)CC3(C)C)[C@](C)(O)C1]

Fucoxanthin is known to be readily decomposed by heating, light irradiation, oxidation, or the like.

As used herein, “manufacture” of a microalgal product refers to a series of processes from the step of preparing a cell to the step of obtaining the microalgal product, some of the steps, or any combination of the steps. Manufacture is used interchangeably with “production”. For example, manufacture of a microalgal product can comprise, but is not limited to, at least one of the step of culturing microalgae, the step of treating the microalgae (e.g., heating), the step of concentrating the microalgae, and the step of drying the microalgae.

As used herein, “culture” is used in the meaning that is commonly used in the art, which refers to an operation for maintaining cells in or on a medium in a viable state. The cell count can be increased, decreased, or maintained during culture. As used herein, “main culture” refers to culture wherein microalgae resulting from the completed culture are used as a raw material for the manufacture of a product. As used herein, “seed culture” refers to any culture other than the main culture. Examples thereof include culture prior to transferring microalgae to a larger scale culture, culture performed under a condition where the cell density does not vary significantly in order to maintain microalgae in a stable state (maintenance culture), culture for changing the state of cells (e.g., change from a dormant state to a stable state (acclimation culture) and change from a stable state to a rapidly growing state), and the like.

As used herein, “concentration” refers to an operation to increase the cell density by means that is not dependent on cell growth (e.g., centrifugation, filtration, removal of medium, etc.). Concentration can also be expressed as the maintenance of the cell density of the microalgae at 3 g/L (dry weight).

As used herein, “amount of stress” is an indicator of producibility of pheophorbide accumulated by any operation that increases the amount of pheophorbide produced in microalgae. The amount of stress applied by an operation to microalgae is defined as the ratio expressed as (amount of existing pheophorbide measured when the operation is performed)/(amount of existing pheophorbide measured when the operation is not performed) when the amount of existing pheophorbide is measured for the NBRC 102809 strain (available from NITE) cultured at normal temperature under the same conditions, other than presence/absence of the operation. The amount of existing pheophorbide of about 30 to 90 mg/100 g (dry weight) can be observed for NBRC 102809 strains to which a particularly large stimulation is not applied. The amount of stress can be predicted from the amount of existing pheophorbide measured under similar conditions.

As used herein, “high amount of stress” refers to an amount of stress where the total amount of stress applied by each operation is greater than or equal to 5.

As used herein, “low amount of stress” refers to an amount of stress where the total amount of stress applied by each operation is less than or equal to 2.

As used herein, the “amount” of an analyte in a sample generally refers to an absolute value reflecting the mass of the analyte that can be detected in a volume of the sample. However, amount is also intended as a relative amount as compared to the amount of another analyte. For example, the amount of an analyte in a sample can be an amount that is greater than a control level or a normal level of an analyte that is generally present in the sample.

As used herein, the term “about” refers to the indicated value plus or minus 10%, unless explicitly noted otherwise.

As used herein, “system” refers to a configuration for executing the method or program of the present disclosure, inherently meaning an architecture or organization for executing an objective. A plurality of elements are systematically configured, affecting each other. In the field of computers, a system refers to the entire configuration of hardware, software, OS, network, and the like. However, it is understood that a computer is not necessarily required to be used in the present disclosure, so that a construct consisting of various configurations are within the scope of system.

### Preferred Embodiments

Preferred embodiments of the present disclosure are described below. Embodiments described below are provided to facilitate the understanding of the present disclosure. It is understood that the scope of the present disclosure should not be limited to the following descriptions. Thus, it is apparent that those skilled in the art can make appropriate modifications within the scope of the present disclosure by referring to the descriptions herein. It is also understood that the following embodiments of the present disclosure can be used independently or as a combination thereof.

(Microalgal Product)

In one aspect, the present disclosure provides a microalgal product. In one embodiment, the microalgal product is a food product or a cosmetic product. The inventors found a method of preparing a microalgal product while suppressing the generation of pheophorbide in microalgae, enabling safe microalgal products (e.g., food products and cosmetic products) to be provided. In one embodiment, the amount of pheophorbide contained in the microalgal product of the present disclosure (% by weight of each component is defined herein as per weight excluding moisture) can be less than or equal to about 1% by weight, less than or equal to about 0.7% by weight, less than or equal to about 0.5% by weight, less than or equal to about 0.2% by weight, less than or equal to about 0.1% by weight, less than or equal to about 0.07% by weight, less than or equal to about 0.05% by weight, less than or equal to about 0.02% by weight, less than or equal to about 0.01% by weight, less than or equal to about 0.007% by weight, less than or equal to about 0.005% by weight, less than or equal to about 0.002% by weight, less than or equal to about 0.001% by weight, less than or equal to about 0.0007% by weight, less than or equal to about 0.0005% by weight, less than or equal to about 0.0002% by weight, less than or equal to about 0.0001% by weight, or the like.

Any microalgae can be used for the microalgal product of the present disclosure. The microalgal product of the present disclosure can comprise either the algal body of microalgae or extract of a microalgal component. In this regard, algal body refers to not only an undamaged cell, but also a cell that is ruptured so that the cellular components are separated. For example, this can refer to a state where a microalgal product contains one of the primary constituent components of an algal cell (e.g., cell wall, cell membrane, protein, lipid, or carbohydrate) at 10% by weight of greater, 5% by weight of greater, 1% by weight of greater, 0.5% by weight of greater, 0.1% by weight of greater, 0.05% by weight of greater, 0.01% by weight of greater, 0.005% by weight of greater, 0.001% by weight of greater, 0.0005% by weight of greater, or 0.0001% by weight of greater. Examples of microalgae that can be used include organisms of the phylum cyanobacteria as well as eukaryotic organisms of the phylum Glaucophyta, Rhodophyta (red algae), Chlorophyta, Cryptophyta (cryptophytes), Haptophyta (haptophytes), Heterokontophyta, Dinophyta (dinoflagellates), Euglenida, and Chlorarachniophyta. For example, microalgae of the phylum Chlorophyta that can be used include the class Trebouxiophyceae. The class Trebouxiophyceae includes the order Chlorellales. The order Chlorellales includes the family Chlorellaceae. The family Chlorellaceae includes the genus Chlorella. For example, microalgae of Euglenida that can be used include the class Euglenophyceae. The class Euglenophyceae includes the order Euglenales. The order Euglenales includes the family Euglenaceae. The family Euglenaceae includes the genus Euglena. For example, microalgae of the phylum cyanobacteria that can be used include the order Oscillatoriales. The order Oscillatoriales includes the genus Arthrospira. For example, microalgae of the phylum Haptophyta (haptophytes) that can be used include the class Haptophyceae. The class Haptophyceae includes the subclasses Pavlovophycidae and Prymnesiophycidae. The subclass Pavlovophycidae includes the order Pavlovales. The order Pavlovales includes the family Pavlovaceae. The family Pavlovaceae includes Diacronema, Exanthemachrysis, Pavlova, and Rebecca. The subclass Prymnesiophycidae includes the order Isochrysidales. The order Isochrysidales includes the genera Isochrysis, Imantonia, Emiliania, Gephyrocapsa, and Reticulofenestra. Isochrysis includes I. galbana, I. litoralis, I. maritima, and Tisochrysis lutea. Emiliania includes E. huxleyi. Gephyrocapsa includes G. oceanica, G. ericsonii, G. muellerae, and G. protohuxleyi. Microalgae of the order Isochrysidales and microalgae of the order Pavlovales can have the same property of producing fucoxanthin and producing EPA at a high level. Thus, microalgae belonging to the order Pavlovales and microalgae belonging to the order Isochrysidales at least have the same issue to be solved in terms of the problem of pheophorbide production that can be problematic in fucoxanthin production in the present disclosure. Therefore, those skilled in the art understand that the issue to be solved is solved in the same manner by the content of the present disclosure in such a context in the present disclosure. A preferred embodiment of the present disclosure can comprise, as the target microalgae, microalgae whose amount of pheophorbide production can be problematic. Examples of such microalgae include, but are not limited to, microalgae of the order Euglenida (e.g., microalgae of the family Euglenaceae and genus Euglena described above), the order Pavlovales (e.g., microalgae of the family Pavlovaceae and genus Pavlova described above), and the order Isochrysidales (e.g., microalgae of the family Isachrysidaceae and genus Isochrysis described above). In one embodiment, the microalgae used are microalgae of the family Pavlovaceae. In one embodiment, the microalgae used are microalgae of the genus Pavlova. In one embodiment, the microalgae used are P. calceolate, P. granifera, P. gyrans, P. lutheri, P. pinguis, or P. salina.

In one embodiment, pheophorbide is reduced in microalgae contained in the microalgal product of the present disclosure. In one embodiment, pheophorbide content of microalgae contained in the microalgal product of the present disclosure can be less than or equal to about 1% by weight, less than or equal to about 0.7% by weight, less than or equal to about 0.5% by weight, less than or equal to about 0.2% by weight, less than or equal to about 0.1% by weight, less than or equal to about 0.07% by weight, less than or equal to about 0.05% by weight, less than or equal to about 0.02% by weight, less than or equal to about 0.01% by weight, less than or equal to about 0.007% by weight, less than or equal to about 0.005% by weight, less than or equal to about 0.002% by weight, less than or equal to about 0.001% by weight, or the like. The pheophorbide content of microalgae contained in a microalgal product can be calculated from (amount of pheophorbide contained in microalgal product)/(amount of microalgae contained in microalgal product).

In one embodiment, microalgae that produce fucoxanthin at a high level (e.g., class Haptophyceae) can be used in the microalgal product of the present disclosure. In one embodiment, an amount of fucoxanthin contained in the microalgal product of the present disclosure (% by weight of each component is defined herein as per weight excluding moisture) can be greater than or equal to about 0.001% by weight, greater than or equal to about 0.002% by weight, greater than or equal to about 0.005% by weight, greater than or equal to about 0.007% by weight, greater than or equal to about 0.01% by weight, greater than or equal to about 0.02% by weight, greater than or equal to about 0.05% by weight, greater than or equal to about 0.07% by weight, greater than or equal to about 0.1% by weight, greater than or equal to about 0.2% by weight, greater than or equal to about 0.5% by weight, greater than or equal to about 0.7% by weight, greater than or equal to about 1% by weight, greater than or equal to about 2% by weight, greater than or equal to about 5% by weight, or the like. In one embodiment, the fucoxanthin content of microalgae contained in the microalgal product of the present disclosure can be greater than or equal to about 0.01% by weight, greater than or equal to about 0.02% by weight, greater than or equal to about 0.05% by weight, greater than or equal to about 0.07% by weight, greater than or equal to about 0.1% by weight, greater than or equal to about 0.2% by weight, greater than or equal to about 0.5% by weight, greater than or equal to about 0.7% by weight, greater than or equal to about 1% by weight, greater than or equal to about 2% by weight, greater than or equal to about 5% by weight, or the like. The fucoxanthin content of microalgae contained in a microalgal product can be calculated from (amount of fucoxanthin contained in microalgal product)/(amount of microalgae contained in microalgal product). Since fucoxanthin is known to have an anti-obesity effect, antidiabetic effect, antioxidant effect, anticancer effect, angiogenesis suppression effect, and the like, the microalgal product of the present disclosure containing a large amount of fucoxanthin is expected to attain such effects.

In one embodiment, microalgae that produce chlorophylls at a high level (e.g., class Haptophyceae) can be used in the microalgal product of the present disclosure. While chlorophylls can produce pheophorbide, the inventors found a method of manufacturing a microalgal product by processing microalgae so that the amount of pheophorbide would not increase, even for microalgae that produce chlorophylls at a high level. Thus, even microalgae that produce chlorophylls at a high level can be suitably used. In one embodiment, the amount of chlorophyll contained in the microalgal product of the present disclosure (% by weight of each component is defined herein as per weight excluding moisture) can be greater than or equal to about 0.001% by weight, greater than or equal to about 0.002% by weight, greater than or equal to about 0.005% by weight, greater than or equal to about 0.007% by weight, greater than or equal to about 0.01% by weight, greater than or equal to about 0.02% by weight, greater than or equal to about 0.05% by weight, greater than or equal to about 0.07% by weight, greater than or equal to about 0.1% by weight, greater than or equal to about 0.2% by weight, greater than or equal to about 0.5% by weight, greater than or equal to about 0.7% by weight, greater than or equal to about 1% by weight, greater than or equal to about 2% by weight, greater than or equal to about 5% by weight, greater than or equal to about 7% by weight, greater than or equal to about 10% by weight, greater than or equal to about 20% by weight, greater than or equal to about 30% by weight, greater than or equal to about 40% by weight, or the like. In one embodiment, the chlorophyll content of microalgae contained in the microalgal product of the present disclosure can be greater than or equal to about 0.01% by weight, greater than or equal to about 0.02% by weight, greater than or equal to about 0.05% by weight, greater than or equal to about 0.07% by weight, greater than or equal to about 0.1% by weight, greater than or equal to about 0.2% by weight, greater than or equal to about 0.5% by weight, greater than or equal to about 0.7% by weight, greater than or equal to about 1% by weight, greater than or equal to about 2% by weight, greater than or equal to about 5% by weight, greater than or equal to about 7% by weight, greater than or equal to about 10% by weight, greater than or equal to about 20% by weight, greater than or equal to about 30% by weight, greater than or equal to about 40% by weight, or the like. The chlorophyll content of microalgae contained in a microalgal product can be calculated from (amount of chlorophyll contained in microalgal product)/(amount of microalgae contained in microalgal product).

In one embodiment, the microalgal product of the present disclosure can be an edible product such as a food product, a feed, a supplement, a food additive, or a beverage, or any edible product. A microalgal product (% by weight of each component is defined herein as per weight excluding moisture), which is a food product, can contain microalgae or a component thereof at about 0.001 to 100% by weight, such as about 0.001% by weight, about 0.002% by weight, about 0.005% by weight, about 0.007% by weight, about 0.01% by weight, about 0.02% by weight, about 0.05% by weight, about 0.07% by weight, about 0.1% by weight, about 0.2% by weight, about 0.5% by weight, about 0.7% by weight, about 1% by weight, about 2% by weight, about 5% by weight, about 7% by weight, about 10% by weight, about 20% by weight, about 50% by weight, about 70% by weight, or about 100% by weight. For example, microalgae of the order Pavlovales can have a property of being soft with no cell walls. Thus, such microalgae do not have an unpleasant mouthfeel upon intake. If the taste or flavor of microalgae is a concern, any suitable flavor masking agent, odor masking agent, or other masking agent can be used concomitantly. The taste or flavor of microalgae can be masked using means such as coating or capsulation. Since pheophorbide of microalgae can be reduced in the present disclosure, a microalgae product (e.g., supplement or food additive) can comprise microalgae at a high concentration such as about 10% by weight or greater. Since microalgae containing a useful component such as fucoxanthin in abundance can attain an effect from a small amount of intake, such microalgae can be used as a supplement and/or food additive.

In one embodiment, the microalgal product of the present disclosure can be any cosmetic product. A microalgal product (% by weight of each component is defined herein as per weight excluding moisture), which is a cosmetic product, can comprise microalgae or a component thereof at about 0.001 to 100% by weight such as about 0.001% by weight, about 0.002% by weight, about 0.005% by weight, about 0.007% by weight, about 0.01% by weight, about 0.02% by weight, about 0.05% by weight, about 0.07% by weight, about 0.1% by weight, about 0.2% by weight, about 0.5% by weight, about 0.7% by weight, about 1% by weight, about 2% by weight, about 5% by weight, about 7% by weight, about 10% by weight, about 20% by weight, about 50% by weight, about 70% by weight, or about 100% by weight. For example, microalgae of the order Pavlovales can have a property of being soft with no cell walls. Thus, such microalgae have little stimulation when applied to the skin. If the odor or the like of microalgae is a concern, any suitable odor masking agent or other masking agent can be used concomitantly, or a component of microalgae can be masked by using means such as coating or capsulation. Since pheophorbide of microalgae can be reduced in the present disclosure, a microalgal cosmetic product can be used safely, even when microalgae are contained at a high concentration such as 10% by weight of greater.

In one embodiment, the microalgal product of the present disclosure is for mammals. In one embodiment, the microalgal product of the present disclosure is for humans (e.g., for human consumption).

In one embodiment, the microalgal product of the present disclosure can be dry or contain moisture. In one embodiment, the amount of moisture in the microalgal product of the present disclosure can be about 0.1 by weight to about 50% by weight, such as about 0.5% by weight, about 1% by weight, about 1.5% by weight, about 2% by weight, about 2.5% by weight, about 3% by weight, about 4% by weight, about 5% by weight, about 7% by weight, about 10% by weight, about 20% by weight, about 30% by weight, about 40% by weight, about 50% by weight, particularly about 2.5% by weight, about 3% by weight, about 4% by weight, or the like. Preservability, processability, or the like of microalgae can be improved by drying. In one embodiment, the microalgal product of the present disclosure in a dry state can comprise one or more of an excipient (dextrin, etc.), desiccant, antioxidant, and deoxygenation agent, which can reduce decomposition of a component (e.g., fucoxanthin) of microalgae. Any desiccant, antioxidant, and deoxygenation agent, which can be added to a food product or enclosed within the packaging of a food product, can be used. In one embodiment, a desiccant, antioxidant, and/or deoxygenation agent can be added to the microalgal product of the present disclosure by placing them in a container such as an air permeable bag. In one embodiment, the microalgal product of the present disclosure (e.g., dried product) can contain an emulsifier, which can promote, for example, an antioxidant to enter microalgal cells. In one embodiment, the microalgal product of the present disclosure (e.g., dried product) can be enclosed in a lightproof container, which can reduce decomposition of a component (e.g., fucoxanthin) of microalgae. The microalgal product of the present disclosure (e.g., dried product) can be stored at low temperature, which can reduce the decomposition of a component (e.g., fucoxanthin) of microalgae.

The microalgal product of the present disclosure can be in any suitable form. In one embodiment, the microalgal product of the present disclosure can be in a form of, for example, a tablet (dry), powder, capsule, cream, frozen product, liquid, or the like, but the form is not limited thereto.

In one embodiment, the microalgal product of the present disclosure can be an oil immersed product. Oil can be any edible oil, such as olive oil, rapeseed oil, perilla oil, flaxseed oil, corn oil, soybean oil, sunflower oil, safflower oil, cottonseed oil, rice oil, argan oil, avocado oil, almond oil, peanut oil, butter, tallow, lard, shortening, margarine, coconut oil, palm oil, coconut oil, or the like. In a specific embodiment, a dried microalgae of the present disclosure is used in an oil immersed product. The mixing ratio of microalgae of the present disclosure:oil in an oil immersed product can be, for example, about 1:100 to 100:1, about 1:50 to 50:1, about 1:20 to 20:1, about 1:10 to 10:1, about 1:5 to 5:1, about 1:75, about 1:50, about 1:25, about 1:20, about 1:15, about 1:10, about 1:7, about 1:5, about 1:2, about 1:1, about 2:1, about 5:1, about 7:1, about 10:1, about 15:1, about 20:1, about 25:1, about 50:1, about 75:1, or about 100:1 in terms of weight. In one embodiment, the oil immersed product of the present disclosure can comprise an antioxidant. Examples of antioxidant include, but are not limited to, vitamin E (tocopherol and tocotrienol), ascorbic acid, β-carotene, vitamin A, lycopene, chlorogenic acid, ellagic acid, lignan, sesamin, curcumin, coumarin, oleocanthal, oleuropein, resveratrol, catechin, anthocyanin, tannin, rutin, isoflavone, nobiletin, lutein, zeaxanthin, canthaxanthin, astaxanthin, β-cryptoxanthin, rubixanthin, and ubiquinol. In one embodiment, the oil immersed product of the present disclosure can be provided in a form emulsified by an emulsifier. In one embodiment, the oil immersed product of the present disclosure can comprise an excipient (dextrin or the like). In one embodiment, the oil immersed product of the present disclosure can be provided while being encapsulated in an edible capsule.

In one embodiment, the microalgal product of the present disclosure can be a frozen product. At low temperatures, decomposition of a component (e.g., fucoxanthin) of microalgae can be reduced. In one embodiment, a frozen product can be, but is not limited to, dairy product free, sherbet, a LACT-ICE (with at least 3% milk solids), an ICE-MILK (with at least 10% milk solids and 3% milk fat), an ICE-CREAM (with at least 15% milk solids and 8% milk fat), ice candy, soft serve ice cream, or the like. An excipient (cyclodextrin, saccharide, etc.), juice (e.g., citrus, grape, apple, peach, etc.), fruit extract, vegetable juice, sweetener, flavoring, coloring agent, antioxidant, thickener, or the like can be added to a frozen product. In one embodiment, a frozen product can be in a sheet-like form or in a cup. Since the microalgae of the present disclosure can have a sea grass flavor, an additive for masking such a flavor (e.g., juice, fruit extract, or flavoring) can be added to a frozen product.

In one embodiment, the microalgal product of the present disclosure (% by weight of each component is defined herein as per weight excluding moisture) can comprise:

about 1 to 5% by weight of the microalgae of the present disclosure and less than or equal to about 0.1% by weight, less than or equal to about 0.07% by weight, less than or equal to about 0.05% by weight, less than or equal to about 0.02% by weight, less than or equal to about 0.01% by weight, less than or equal to about 0.007% by weight, less than or equal to about 0.005% by weight, less than or equal to about 0.002% by weight, or less than or equal to about 0.001% by weight of pheophorbide,

about 5 to 10% by weight of the microalgae of the present disclosure and less than or equal to about 0.2% by weight, less than or equal to about 0.15% by weight, less than or equal to about 0.1% by weight, less than or equal to about 0.05% by weight, less than or equal to about 0.02% by weight, less than or equal to about 0.015% by weight, less than or equal to about 0.01% by weight, less than or equal to about 0.005% by weight, or less than or equal to about 0.002% by weight of pheophorbide,

about 10 to 20% by weight of the microalgae of the present disclosure and less than or equal to about 0.5% by weight, less than or equal to about 0.2% by weight, less than or equal to about 0.1% by weight, less than or equal to about 0.05% by weight, less than or equal to about 0.02% by weight, less than or equal to about 0.015% by weight, less than or equal to about 0.01% by weight, less than or equal to about 0.007% by weight, or less than or equal to about 0.005% by weight of pheophorbide,

about 20 to 50% by weight of the microalgae of the present disclosure and less than or equal to about 1% by weight, less than or equal to about 0.7% by weight, less than or equal to about 0.5% by weight, less than or equal to about 0.2% by weight, less than or equal to about 0.1% by weight, less than or equal to about 0.07% by weight, less than or equal to about 0.05% by weight, less than or equal to about 0.02% by weight, less than or equal to about 0.01% by weight, or less than or equal to about 0.005% by weight of pheophorbide, or

about 50 to 100% by weight of the microalgae of the present disclosure and less than or equal to about 2% by weight, less than or equal to about 1.5% by weight, less than or equal to about 1% by weight, less than or equal to about 0.5% by weight, less than or equal to about 0.2% by weight, less than or equal to about 0.15% by weight, less than or equal to about 0.1% by weight, less than or equal to about 0.07% by weight, less than or equal to about 0.05% by weight, less than or equal to about 0.02% by weight, or less than or equal to about 0.01% by weight of pheophorbide.

(Manufacturing Method of Microalgal Product)

In one aspect, the present disclosure provides a method of manufacturing a microalgal product. The manufacturing method comprises at least one of the step of culturing microalgae, the step of treating the microalgae, the step of concentrating the microalgae, the step of drying the microalgae, and the step of separating a component of the microalgae. Any microalgae that can be used in the microalgal product of the present disclosure described above can be used in this manufacturing method. The manufacturing method can also be performed to achieve any state (e.g., pheophorbide, fucoxanthin, and/or chlorophyll content) of microalgae contained in the microalgal product of the present disclosure described above.

One feature of the present disclosure is comprising subjecting microalgae to treatment (e.g., heating) for inactivating a chlorophyllase under a condition for controlling an amount of stress in any step of a method of manufacturing a microalgal product. In various embodiments of the present disclosure, the condition for controlling an amount of stress of microalgae can be any condition. Examples thereof include a condition under which microalgae are not concentrated, condition for maintaining microalgae at or below a certain cell density, a condition for limiting the pressure applied to a cell upon concentration (e.g., strength of G upon centrifugation) and/or the time length thereof (e.g., time length of centrifugation) to the extent this does not result in an adverse effect, a condition for reducing physical and chemical damages to a cell from concentration by administration of an additive (e.g., flocculant or coagulant), and the like. In one embodiment, step (A) can comprise a step of measuring an amount of stress. Controlling of the amount of stress for suppressing pheophorbide can be achieved, for example, by maintaining a density of microalgae at a low level and/or not significantly concentrating microalgae. In one embodiment, an amount of stress applied to microalgae from after culture to treatment for inactivating a chlorophyllase can be maintained at or below a predetermined value by maintaining a density of microalgae at or below a predetermined value and/or not concentrating microalgae by a predetermined factor or greater. The predetermined value of the density and/or the predetermined factor of concentration therein can be determined based on an increase in pheophorbide upon concentrating the microalgae of interest.

Controlling of the amount of stress can be materialized by “suppressing the stimulation level prior to heating as much as possible”. For example, an amount of stimulation applied to microalgae such as Pavlova can be specified, or the state of stimulated Pavlova can be specified. Examples thereof which are highly visible include the outer appearance, i.e., the shape is distorted by a centrifugal force or cell is damaged because Pavlova has no cell walls and is soft.

In particular, the present disclosure is characterized by providing a method for manufacturing a microalgal product, comprising:
(A) a step of maintaining microalgae under a condition where an amount of stress applied to the microalgae from after culture to step (B) is controlled to a predetermined value or less, wherein the step maintains a density of the microalgae at or below a predetermined value and/or does not concentrate the microalgae by a predetermined factor or greater; and
(B) a step of subjecting the microalgae to treatment for inactivating chlorophyllase.

The inventors unexpectedly found that harmful pheophorbide is generated when microalgae are exposed to a stress load. After looking for a method for avoiding pheophorbide generation, the inventors found that subjecting microalgae to treatment for inactivating a chlorophyllase can suppress subsequent increase in pheophorbide. However, when a large amount of pheophorbide is already generated before the chlorophyllase inactivation treatment, the effect of suppressing pheophorbide by the chlorophyllase inactivation treatment was limited. When studies were repeatedly conducted to achieve a more effective pheophorbide suppression in microalgae that can produce a large amount of chlorophylls that are the source of pheophorbide, it was found that it is important to avoid a stress load, especially to avoid a stress load from a high density and concentration process, during the period after culture and before chlorophyllase inactivation treatment. For “hard” microalgae with cell walls such as Chlorella, a high density or concentration process is not considered as a factor that stresses cells. This finding is expected to be unique to “soft” microalgae of the order Pavlovales. For this reason, the need to pay attention to cell density and concentration process during a period from after culturing to before chlorophyllase inactivation treatment is itself a problem that was not previously recognized. Such a problem is new, but those skilled in the art can readily determine the limit for the density and concentration factor that are required for achieving a desired pheophorbide suppression effect for the microalgae used. For example, the acceptable density and concentration factor can be determined by experimentally verifying how much the amount of pheophorbide increases upon concentrating microalgae.

In one embodiment, the method of manufacturing a microalgal product of the present disclosure comprises a step of culturing microalgae. In one embodiment, the step of culturing can be further divided into a step of performing a seed culture, a step of performing a main culture, and the like. In one embodiment, the seed culture can comprise a plurality of culturing stages (e.g., any combination of a culturing stage using a test tube (about 100 mL), a culturing stage using a plastic bottle, flask, or medium bottle (about 1 L or less), a culturing stage using the photobioreactor of the present disclosure (about 5 L), a culturing stage using 10 to 20 photobioreactors of the present disclosure with about a 5 L capacity or 2 to 4 photobioreactors of the present disclosure with about a 25 L capacity (about 50 to 100 L), and a culturing stage using a larger scale photobioreactor (about 1000 L or greater)). Unless specifically noted otherwise, the culture conditions described below can be applied to any type of culture. The conditions (e.g., temperature, pH, agitation condition, light irradiation condition, and medium composition) for the step of culturing microalgae can each be determined appropriately. In one embodiment, culture of microalgae can comprise a plurality of stages (e.g., seed culture and main culture, indoor contamination-free culture and outdoor high speed growth culture, acclimation culture and main culture, or the like). The step of culturing microalgae can comprise growing the microalgae to a density of 1.5 g/L (dry weight) or 1.7 g/L (dry weight) or greater.

In one embodiment, microalgae can be cultured at a temperature of about 0° C. to 80° C., more specifically at about 20° C. to 30° C. The upper limit of a suitable temperature can be 80° C., 70° C., 60° C., 50° C., 40° C., 30° C., 20° C., or the like. The lower limit can be 0° C., 5° C., 10° C., 15° C., 20° C., 25° C., 30° C., or the like. Any combination thereof can be employed as a suitable temperature range, as long as there is no inconsistency. Any culturing temperature can be used, as long as microalgae do not die. The culturing temperature does not need to be constant. Especially when a culture vessel is installed outdoors, the temperature does not need to be strictly managed. It is preferable to subject microalgae to a temperature at which the microalgae can suitably survive/grow at least for a portion of the culture period. If the temperature increases too much due to direct sun light or the like, the temperature can be reduced by any cooling means (e.g., water cooling). If, for example, microalgae are haptophytes, the microalgae can suitably grow at a temperature of about 25 to 30° C.

In one embodiment, microalgae can be cultured at a pH of about 2 to 13. The upper limit of a suitable pH can be a pH of 13, 12, 11, 10, 9, 8.5, 8, 7.5, 7, 6, or the like. The lower limit can be a pH of 2, 3, 4, 5, 6, 6.5, 7, 7.5, 8, or the like. Any combination thereof can be employed as a suitable range of pH, as long as there is no inconsistency. Any pH can be used, as long as microalgae do not die. While the suitable pH can vary by type of microalgae, those skilled in the art can readily determine a pH that is suitable for the microalgae used. It is preferable that pH does not change rapidly during culture. The change in pH can be controlled by using any suitable buffer (e.g., carbon dioxide, an amine compound, or the like). If, for example, microalgae are haptophytes, the microalgae can suitably grow in a weakly alkaline environment with a pH of about 8.

In one embodiment, microalgae can, but does not need to be subjected to an agitating condition during culture. Examples of agitation means include, but are not limited to, aeration agitation, mechanical agitation (paddle agitation, etc.), flowing water agitation (e.g., using a pump), agitation by shaking a culture vessel, and the like. Microalgae can be damaged depending on the agitation means. Euglena, haptophytes, and the like in particular are relatively soft without cell walls, so that it can be preferable to avoid vigorous agitation that would disrupt cells in culturing.

In one embodiment, microalgae can be cultured under light irradiation during at least a portion of the culture period. While it varies by the type of microalgae, the growth rate of microalgae can improve with a greater amount of light irradiated, to the extent that the microalgae are not damaged. Depending on the microalgae, non-constant light irradiation can be preferable. A specific wavelength region can be selectively irradiated. When microalgae are cultured outdoors, it can be advantageous to utilize natural light. Even if microalgae are cultured outdoors and only natural light is utilized as a light source, the amount of light per cell of microalgae can be controlled by adjusting the depth of a culture vessel or the diameter of a photobioreactor. Especially when haptophytes with a large amount of photosynthetic pigments or the like are grown, it can be advantageous to irradiate a large amount of light such as natural light. The amount of light energy that can be used can be, for example, about 30 μmol m⁻²s^{-1 1} to about 3000 μmol m⁻²s⁻¹ or about 30 μmol m⁻²s⁻¹ to about 1500 μmol m⁻²s⁻¹, or preferably about 50 μmol m⁻²s⁻¹ to about 300 μmol m⁻²s⁻¹. When microalgae are for example haptophytes, the microalgae can be suitably grown with light energy at about 100 μmol m⁻²s⁻¹ to about 150 μmol m⁻²s⁻¹.

Any suitable composition can be used for a medium used for culturing microalgae in accordance with the type of microalgae. Representative examples of components that can be contained in a medium include inorganic salts (e.g., potassium salt, sodium salt, calcium salt, and magnesium salt), saccharides (e.g., glucose), organic salts, nitrogen sources (nitric acid salt, ammonium salt, etc.), phosphorous sources (inorganic phosphorous, phosphorous salt, etc.), and the like, but the medium can also contain other components. Since nitrogen sources, phosphorous sources, or the like can be consumed with the growth of microalgae, they can be added when appropriate. If a carbon source (e.g., carbon dioxide) is added, the carbon source can be utilized by microalgae. When, for example, haptophytes are cultured, many haptophytes inhabit the saltwater to brackish water region, so that a medium with a composition that is close to that of seawater to brackish water (e.g., medium comprising about 50 to 75% of salts in seawater) or a medium with an osmotic pressure that is close to that of seawater to brackish water can be used.

Increasing the microalgal density in the culturing step of the manufacturing method of the present disclosure is preferable for improving the efficiency in culture. For example, culture can be performed up to a density of at least 0.01 g/L, at least 0.02 g/L, at least 0.05 g/L, at least 0.07 g/L, at least 0.1 g/L, at least 0.2 g/L, at least 0.5 g/L, at least 0.7 g/L, at least 1 g/L, at least 1.5 g/L, at least 2 g/L, at least 2.5 g/L, at least 3 g/L, at least 3.5 g/L, at least 4 g/L, at least 4.5 g/L, at least 5 g/L, at least 5.5 g/L, at least 6 g/L, at least 7 g/L, at least 8 g/L, at least 9 g/L, at least 10 g/L, at least 20 g/L, at least 50 g/L, or at least 100 g/L in terms of dry weight of microalgae. In particular, microalgae (e.g., haptophytes) can be cultured to a high density of 2 g/L or greater if the apparatus of the present disclosure described in detail below is used. The culturing period can continue until a microalgal density of interest is achieved, or continue for a predetermined culturing period that is specified, or continue indefinitely as in maintenance culture or the like.

In one embodiment, the method of manufacturing a microalgal product of the present disclosure comprises the step of treating microalgae. In one embodiment, the treatment is treatment for inactivating a chlorophyllase. Generation of pheophorbide can be suppressed by inactivating a chlorophyllase. Examples of treatment for inactivating a chlorophyllase include, but are not limited to, heating, any known protein denaturation treatment (temperature load (low temperature or high temperature), treatment with an agent (alcohol, strong acid, strong base, or other denaturant), or irradiation of radiation (ultraviolet rays, gamma rays, etc.)), and the like. Treatment for inactivating a chlorophyllase (e.g., heating) can be applied under any suitable condition (means, time, etc.) for inactivating a chlorophyllase, but a condition under which microalgae are not destroyed and/or a useful component of the microalgae is not destroyed can be preferably applied. For instance, to be able to produce fucoxanthin, haptophytes can be preferably treated under a condition where decomposition of fucoxanthin is low, e.g., decrease in fucoxanthin after treatment in comparison to before treatment is less than 0.01%, less than 0.02%, less than 0.05%, less than 0.07%, less than 0.1%, less than 0.2%, less than 0.5%, less than 0.7%, less than 1%, less than 2%, less than 3%, less than 4%, less than 5%, less than 6%, less than 7%, less than 8%, less than 9%, less than 10%, less than 15%, less than 20%, less than 25%, less than 30%, less than 35%, less than 40%, less than 45%, less than 50%, less than 60%, less than 70%, or less than 80%. Treatment for inactivating a chlorophyllase is preferably performed under a condition where an amount of stress is controlled. It is preferable to perform the treatment on microalgae to which the amount of stress applied prior to the treatment is not high. When treatment for inactivating a chlorophyllase is applied to microalgae to which the amount of stress applied is high, a large amount of pheophorbide is possibly already produced, so that a sufficient effect of suppressing pheophorbide by inactivating a chlorophyllase may not be attained. In one embodiment, the amount of stress applied to microalgae prior to treatment for inactivating a chlorophyllase is less than or equal to 1000, less than or equal to 700, less than or equal to 500, less than or equal to 200, less than or equal to 100, less than or equal to 90, less than or equal to 80, less than or equal to 70, less than or equal to 60, less than or equal to 50, less than or equal to 45, less than or equal to 40, less than or equal to 35, less than or equal to 30, less than or equal to 25, less than or equal to 20, less than or equal to 15, less than or equal to 10, less than or equal to 9, less than or equal to 8, less than or equal to 7, less than or equal to 6, less than or equal to 5, less than or equal to 4.5, less than or equal to 4, less than or equal to 3.5, less than or equal to 3, less than or equal to 2.5, less than or equal to 2, less than or equal to 1.5, or less than or equal to 1.2. In one embodiment, the step of treating microalgae comprises killing microalgae and/or other microorganisms. When a microalgal product is provided as a food product or a food additive, a product that is free of living organisms can be more readily used. Examples of such treatment for killing microalgae and/or other microorganisms include, but are not limited to, heating, irradiation of radiation, and the like.

In one embodiment, treatment for inactivating a chlorophyllase is heating, which can be heating at about 50° C. to 200° C., such as about 50° C., about 60° C., about 70° C., about 80° C., about 85° C., about 90° C., about 95° C., about 97° C., about 100° C., about 102° C., about 105° C., about 107° C., about 110° C., about 120° C., about 130° C., about 140° C., about 150° C., about 160° C., about 170° C., about 180° C., about 190° C., about 200° C., or the like. The period of heating can be about 10 seconds to 20 hours, such as about 10 seconds, about 30 seconds, about 1 minute, about 2 minutes, about 5 minutes, about 7 minutes, about 10 minutes, about 15 minutes, about 20 minutes, about 25 minutes, about 30 minutes, about 40 minutes, about 50 minutes, about 1 hour, about 1.5 hours, about 2 hours, about 2.5 hours, about 3 hours, about 4 hours, about 5 hours, about 7 hours, about 10 hours, about 20 hours, or the like.

In one embodiment, the density of microalgae upon treatment for inactivating a chlorophyllase can be about 0.01 to 100 g/L, such as less than or equal to about 100 g/L, less than or equal to about 70 g/L, less than or equal to about 50 g/L, less than or equal to about 40 g/L, less than or equal to about 30 g/L, less than or equal to about 20 g/L, less than or equal to about 15 g/L, less than or equal to about 10 g/L, less than or equal to about 7 g/L, less than or equal to about 5 g/L, less than or equal to about 4 g/L, less than or equal to about 3 g/L, less than or equal to about 2 g/L, less than or equal to about 1 g/L, less than or equal to about 0.5 g/L, or less than or equal to about 0.1 g/L, and greater than or equal to about 0.01 g/L, greater than or equal to about 0.05 g/L, greater than or equal to about 0.1 g/L, greater than or equal to about 0.2 g/L, greater than or equal to about 0.5 g/L, greater than or equal to about 0.7 g/L, greater than or equal to about 1 g/L, greater than or equal to about 2 g/L, greater than or equal to about 3 g/L, greater than or equal to about 4 g/L, greater than or equal to about 5 g/L, greater than or equal to about 7 g/L, or greater than or equal to about 10 g/L in terms of dry weight. When the density of microalgae exceeds for example 10 g/L, the overall inactivation of a chlorophyllase can be insufficient. In one embodiment, microalgae can be subjected to treatment that does not significantly increase the amount of stress from after culture to treatment for inactivating a chlorophyllase. Examples of such treatment include light membrane concentration (1.5-fold concentration, 2-fold concentration, 3-fold concentration, etc.) and the like. In one embodiment, microalgae are not concentrated to the concentration described above from after culture to treatment for inactivating a chlorophyllase. In one embodiment, microalgae are not diluted from after culture to treatment for inactivating a chlorophyllase.

In one embodiment, microalgae are not subjected to high degree of centrifugation before and/or during treatment for inactivating a chlorophyllase. Microalgae are not exposed to gravitational acceleration of, for example, greater than or equal to 50 G, greater than or equal to 100 G, greater than or equal to 200 G, greater than or equal to 500 G, greater than or equal to 700 G, greater than or equal to 1000 G, greater than or equal to 1500 G, greater than or equal to 2000 G, greater than or equal to 2500 G, greater than or equal to 3000 G, greater than or equal to 3500 G, greater than or equal to 4000 G, greater than or equal to 4500 G, greater than or equal to 5000 G, greater than or equal to 6000 G, greater than or equal to 7000 G, greater than or equal to 8000 G, greater than or equal to 9000 G, or greater than or equal to 10000 G, and are not subjected to centrifugation for a period of, for example, about 10 seconds or longer, about 30 seconds or longer, about 1 minute or longer, about 2 minutes or longer, about 5 minutes or longer, about 7 minutes or longer, about 10 minutes or longer, about 15 minutes or longer, about 20 minutes or longer, about 25 minutes or longer, about 30 minutes or longer, about 40 minutes or longer, about 50 minutes or longer, about 1 hour or longer, about 1.5 hours or longer, about 2 hours or longer, about 2.5 hours or longer, about 3 hours or longer, about 4 hours or longer, about 5 hours or longer, about 7 hours or longer, about 10 hours or longer, or about 20 hours or longer.

In one embodiment, the method of manufacturing a microalgal product of the present disclosure comprises the step of concentrating microalgae. Microalgae can be concentrated using any suitable means that is known in the art. Examples thereof include, but are not limited to, centrifugation, filtering, removal of a medium (evaporation etc.), use of a coagulant or flocculant, and the like. A concentration process can increase the amount of stress on microalgae. Since Euglena, the order Pavlovales, and the like in particular are relatively soft without cell walls, pheophorbide production can increase due to a concentration process. The problem to be solved of increased pheophorbide due to concentration of cells, for microalgae of the order Pavlovales or Euglena without cell walls whose pheophorbide production would increase with a concentration process, was first discovered in the present disclosure. For example, the amount of chlorophylls a+b of Chlorella/Chlamydomonas is dependent on the culture condition/timing, but is often about 25 mg per 1 g of dry algal body. Meanwhile, it was found that the amount is unexpectedly high at 35.3 mg per 1 g of dry algal body for Pavlova used in the Examples. Thus, the present disclosure addresses a problem to be solved that was not envisioned in conventional methods involving concentrating microalgae, and provides means for solving such a problem.

In one embodiment, the step of concentrating microalgae is not performed prior to treatment for inactivating a chlorophyllase. When a medium comprising (->containing?) unconcentrated microalgae is subjected to treatment for inactivating a chlorophyllase, more reagents or energy may be needed and can result in a higher degree of environmental impact relative to cases using concentrated microalgae. However, the inventors found a culturing method that can grow microalgae (e.g., haptophytes) to a high density of 2 g/L of greater (e.g., method using the culturing apparatus of the present disclosure described in detail below). Thus, the environmental impact was able to be minimized even when subjecting microalgae to treatment for inactivating a chlorophyllase without concentration.

In one embodiment, the microalgae of the present disclosure are not concentrated 1000-fold or more, 900-fold or more, 800-fold or more, 700-fold or more, 600-fold or more, 500-fold or more, 400-fold or more, 300-fold or more, 200-fold or more, 150-fold or more, 100-fold or more, 90-fold or more, 80-fold or more, 70-fold or more, 60-fold or more, 50-fold or more, 40-fold or more, 30-fold or more, 20-fold or more, 15-fold or more, 10-fold or more, 9-fold or more, 8-fold or more, 7-fold or more, 6-fold or more, 5-fold or more, 4-fold or more, 3-fold or more, 2-fold or more, or 1.5-fold or more, or are not subjected to such a concentration process from after culture to before treatment for inactivating a chlorophyllase.

After treatment for inactivating a chlorophyllase, it is understood that pheophorbide does not increase even if stress is applied to microalgae, so that a concentration process can be performed. In one embodiment, the microalgae of the present disclosure are, after treatment for inactivating a chlorophyllase, concentrated 1000-fold or more, 900-fold or more, 800-fold or more, 700-fold or more, 600-fold or more, 500-fold or more, 400-fold or more, 300-fold or more, 200-fold or more, 150-fold or more, 100-fold or more, 90-fold or more, 80-fold or more, 70-fold or more, 60-fold or more, 50-fold or more, 40-fold or more, 30-fold or more, 20-fold or more, 15-fold or more, 10-fold or more, 9-fold or more, 8-fold or more, 7-fold or more, 6-fold or more, 5-fold or more, 4-fold or more, 3-fold or more, 2-fold or more, or 1.5-fold or more, or subjected to such a concentration process. In one embodiment, microalgae are, after treatment for inactivating a chlorophyllase, concentrated to about 10 g/L or greater, about 20 g/L or greater, about 50 g/L or greater, about 70 g/L or greater, about 100 g/L or greater, about 150 g/L or greater, about 200 g/L or greater, about 300 g/L or greater, about 400 g/L or greater, or about 500 g/L or greater in terms of dry weight, or subjected to such a concentration process.

In one embodiment, the method of manufacturing a microalgal product of the present disclosure comprises the step of drying microalgae. The step can dry microalgae so as to achieve the moisture content of the microalgal product of the present disclosure described above.

In one embodiment, the method of manufacturing a microalgal product of the present disclosure comprises the step of separating a component of microalgae. The algal body of microalgae itself can be useful, but a specific component can also be useful. For this reason, a specific component contained in microalgae can be separated from other microalgal components to increase the concentration of the specific component. In another embodiment, a specific component (harmful component, etc.) can be separated and removed from microalgae. For example, the inventors have found that Pavlova, i.e., haptophytes, contains a large amount of fucoxanthin, so that fucoxanthin can be separated/purified as the microalgal product of the present disclosure.

In one embodiment, the microalgae used in the manufacturing method of the present disclosure can be microalgae that produce chlorophylls at a high level. For example, the microalgae can be microalgae that produce chlorophylls at 0.1 mg/g or more, 0.2 mg/g or more, 0.5 mg/g or more, 0.7 mg/g or more, 1 mg/g or more, 2 mg/g or more, 5 mg/g or more, 7 mg/g or more, 10 mg/g or more, 15 mg/g or more, 20 mg/g or more, 25 mg/g or more, 30 mg/g or more, 40 mg/g or more, 50 mg/g or more, 70 mg/g or more, or 100 mg/g in terms of the dry weight of algal body upon completion of the culturing step. In particular, microalgae that produce chlorophylls at 30 mg/g or more in terms of the dry weight of algal body upon completion of the culturing step can be a microalgae that produce chlorophylls at a high level. Since chlorophylls can generate pheophorbide, microalgae that produce chlorophylls at a high level can have the amount of pheophorbide reduced more significantly by the manufacturing method of the present disclosure. Thus, such microalgae can be encompassed by the target microalgae of the present disclosure.

In one embodiment, the manufacturing method of the present disclosure (e.g., method of manufacturing an oil immersed product) can comprise the step of desalinating a microalgal concentrate. In one embodiment, the desalination step can be performed by adding, to a microalgal concentrate prepared by the method of the present disclosure, water in the amount that is, for example, about 1 to 100-fold, about 2 to 50-fold, about 5 to 20-fold, or about 10-fold, agitating for, for example, about 10 minutes to 5 hours, and then centrifuging.

In one embodiment, the manufacturing method of the present disclosure can, but does not need to comprise the step of drying a microalgal concentrate. Especially when preparing a product containing a large amount of moisture such as dressing or vegetable juice, the method does not need to comprise the drying step. In one embodiment, the drying step can comprise spray drying a microalgal concentrate prepared by the method of the present disclosure. In one embodiment, the drying step can be performed in the presence of one or more of an excipient, an emulsifier, and an antioxidant. An excipient can prevent a component (e.g., fucoxanthin) of microalgae from contacting air to reduce decomposition of the component. An antioxidant can reduce decomposition of a component (e.g., fucoxanthin) of microalgae, and further promote reduction in decomposition of components within microalgal cells in combination with an emulsifier.

In one embodiment, the manufacturing method of the present disclosure can comprise the step of freezing a microalgal concentrate. In one embodiment, the freezing step can comprise freezing (e.g., at or below −40° C.) a microalgal concentrate prepared by the method of the present disclosure, or enclosing (preferably sealing/vacuum packing) the concentrate in a bag (e.g., nylon bag or aluminum bag) that can withstand sterilization by boiling (80 to 100° C.) or retort sterilization and freezing the bag at a low temperature (e.g., at or below −40° C.). In one embodiment, a microalgal concentrate can be enclosed in a bag after shaping and freezing. In one embodiment, the freezing step can comprise quick-freezing (e.g., exposing a microalgal concentrate directly to a low temperature environment). Examples of the amount of microalgal concentrate enclosed in one bag include, but are not limited to, 1 ml, 3 mL, 5 mL, 10 mL, 50 mL, 100 mL, 200 mL, 1 L, 2 L, 3 L, 5 L, 8 L, 10 L, 15 L, 20 L, and the like. In one embodiment, the concentrate can be degassed and infused upon enclosure, or a frozen item can be vacuum packed.

(Apparatus for Culturing Microalgae)

In one aspect, the present disclosure provides an apparatus for culturing microalgae. In one embodiment, the apparatus comprises: at least two culturing sections having a wall made of a transparent material; an upper linking section for linking upper portions of the at least culturing sections with one another; a lower linking section for linking lower portions of the at least culturing sections with one another; and at least one bubble generation device installed in at least one, but not all of the at least two culturing sections; wherein the at least two culturing sections, the upper linking section, and the lower linking section are configured to fluid communicably enclose a medium, and the apparatus is installed so that the upper linking section is further away from an installation floor than the lower linking section is. Since the at least two culturing sections, the upper linking section, and the lower linking section are in fluid communication by the medium, a flow can be generated so that the medium circulates the entire apparatus by generating bubbles. This can efficiently achieve a gently agitated state. In a preferred embodiment, the apparatus of the present disclosure does not have a power source for agitation other than a bubble generation device. Since haptophytes for example can suitably grow under a water flowing condition, use of such an apparatus can be suitable. Further, the apparatus can be utilized while suppressing the water volume with a single control system. In one embodiment, the apparatus of the present disclosure can comprise a plurality of repeating units (e.g., one culturing section+one upper linking section+one lower linking section, two culturing sections+one upper linking section+one lower linking section, or the like) that are linked to one another. In such an embodiment, the volume of a medium constituting one continuous system can be readily changed by adjusting the number of repeating units. The variation in the medium environment can be smaller with a greater medium volume. The apparatus of the present disclosure is a vertical apparatus that generates a water flow primary in the up-down direction. It can be possible to grow microalgae to a higher density relative to a horizontal apparatus in view of factors such as efficient use of light or suitable agitation condition.

In one embodiment, a culturing section can have an elongated tubular shape. In one embodiment, the outer diameter of the culturing section can be about 10 mm to about 1000 mm, such as about 10 mm, about 30 mm, about 50 mm, about 70 mm, about 100 mm, about 150 mm, about 200 mm, about 250 mm, about 300 mm, about 400 mm, about 500 mm, about 700 mm, about 1000 mm, or any value between such values. Since the amount of light received per volume of culturing section increases with a smaller culturing section diameter, this can be more suitable for growing microalgae. In one embodiment, the inner diameter of a culturing section can be about 5 mm to about 1000 mm, such as about 5 mm, about 7 mm, about 10 mm, about 30 mm, about 50 mm, about 70 mm, about 100 mm, about 150 mm, about 200 mm, about 250 mm, about 300 mm, about 400 mm, about 500 mm, about 700 mm, or about 1000 mm. In one embodiment, the length of a culturing section can be 10 cm to 1000 cm, such as about 10 cm, about 20 cm, about 50 cm, about 70 cm, about 100 cm, about 150 cm, about 200 cm, about 250 cm, about 300 cm, about 400 cm, about 500 cm, about 1000 cm, or any value between such values. Examples of the transparent material of a wall of a culturing section include, but are not limited to, acrylic material, glass material, and polyethylene material. Any material that allows transmission of a specific wavelength can be used. For example for the OPMS 30543 strain, wavelengths around 430 nm and 680 nm can be useful for photosynthesis, so that materials with high transmittance of light with such wavelengths are preferable.

In one embodiment, the apparatus is configured so that the light receiving area per 1 L is at least 10 cm²/L, at least 20 cm²/L, at least 50 cm²/L, at least 70 cm²/L, at least 100 cm²/L, at least 150 cm²/L, at least 200 cm²/L, at least 250 cm²/L, at least 300 cm²/L, at least 350 cm²/L, at least 400 cm²/L, at least 450 cm²/L, at least 500 cm²/L, at least 550 cm²/L, at least 600 cm²/L, at least 650 cm²/L, at least 700 cm²/L, at least 750 cm²/L, at least 800 cm²/L, at least 900 cm²/L, or at least 1000 cm²/L. In one embodiment, the apparatus can have a configuration in which all culturing sections receive approximately an equal amount of light. For example, in one embodiment, culturing sections are separate portions in a relationship wherein none includes any other culturing section. An apparatus of the present disclosure, if configured so that separate culturing sections do not block light by one another (e.g., not in contact), can be advantageous because the amount of light received increases.

In one embodiment, a linking section can be, but does not need to be made of a transparent material. If a linking section is not made of a transparent material, the efficiency of receiving light of the entire apparatus can be improved by reducing the volume within the linking section. In one embodiment, a linking section can have a shape that does not suppress the flow of a medium (e.g., shape that is not excessively thinner relative to a culturing section), but the linking section can comprise a structure that can appropriately suppress the flow of medium (valve, etc.).

In one embodiment, a hole can be provided on an upper linking section. For example, a tube such as an air introducing tube, CO₂ introducing tube, pH meter, or deaeration tube, a meter, a chord, or the like can be inserted through such a hole.

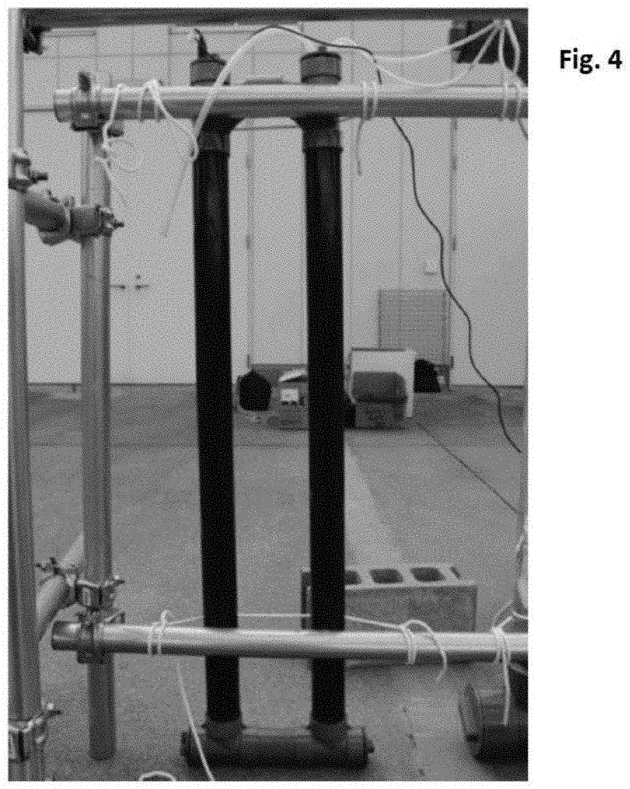

In one embodiment, a bubble generation device can be an air stone or a gas introducing hole provided on the bottom part of a culturing section. In one embodiment, a bubble generation device is installed at a location that is closer to a lower linking section than to an upper linking section. Generating bubbles at a deep part of a medium can improve the efficiency of agitation due to ascending bubbles generating a flow of a medium. For example, water pressure is low for a water depth up to 2 m, so that gas can be readily introduced with an air stone. In one embodiment, a bubble generation device can be a plurality of bubble generation devices each independently introducing a respective type of a plurality of types of gases (e.g., air and carbon dioxide). A bubble generation device preferably has a size that does not inhibit a water flow in the apparatus. The device can have a diameter that is, for example, less than or equal to 80%, less than or equal to 70%, less than or equal to 60%, less than or equal to 50%, less than or equal to 40%, less than or equal to 30%, less than or equal to 20%, or less than or equal to 10% of the inner diameter of a culturing section. In the culturing instrument shown in FIG. 4, the air stone, which is a bubble generation device, has a diameter that is about 28% of the inner diameter of a culturing section. It can be preferable to generate a flow in a certain direction. Thus, if the apparatus of the present disclosure comprises for example four culturing sections, bubble generation devices can be installed at regular positions, such as only in two culturing sections at both ends or only in two center culturing sections.

In one embodiment, an apparatus for culturing microalgae is configured so that a medium contacts with outside air only through a filter and a bubble generation device. In one embodiment, microalgae (e.g., haptophytes) that are vulnerable to contamination (e.g., bacterial contamination) can be stably cultured, and cultured with little contamination by configuring an apparatus so that the inside of the apparatus is independent from the external environment of the apparatus. In one embodiment, an apparatus can be equipped with a spigot for collecting water.

In one embodiment, an apparatus can comprise a sensor, e.g., pH meter, temperature gauge, pressure gauge, oxygen gauge, water hardness gauge, and ammonium gauge. The apparatus can be configured so that this culturing apparatus or another apparatus for manufacturing a microalgal product is controlled based on an input signal from a sensor.

In one embodiment, standard products for water service materials can be primarily used for manufacture, and improvements can be made in the plug of a pH meter, selection of air stone, location of aeration, the shape thereof, or the like by referring to the descriptions herein for simplicity or cost reduction.

Since the apparatus for culturing microalgae described above can be capable of culturing microalgae with little contamination, the apparatus can be used suitably, especially for seed culture prior to main culture.

The thickness of a tube used in the present disclosure can be adjusted, as long as the outer diameter of an acrylic tube or glass tube meets the specification for water service material. For example, a tube prepared according to the specification of 50A and 100A can be used.

(System for Manufacturing Microalgal Product)

In one aspect, the present disclosure provides a system for manufacturing a microalgal product (e.g., food product). The system can comprise any suitable means for performing the method of manufacturing a microalgal product described above. In one embodiment, the present disclosure provides a system comprising a culture vessel and a treatment section for applying treatment for inactivating a chlorophyllase, wherein a section from the culturing section to the treatment section is configured to be able to control an amount of stress applied to microalgae.

A pump (flow rate varying unit) can be installed at any part of the system of the present disclosure. A pump can be, for example, a syringe pump, plunger pump, piston pump, or roller pump. The flow rate, pressure, or the like can be adjusted with a pump.

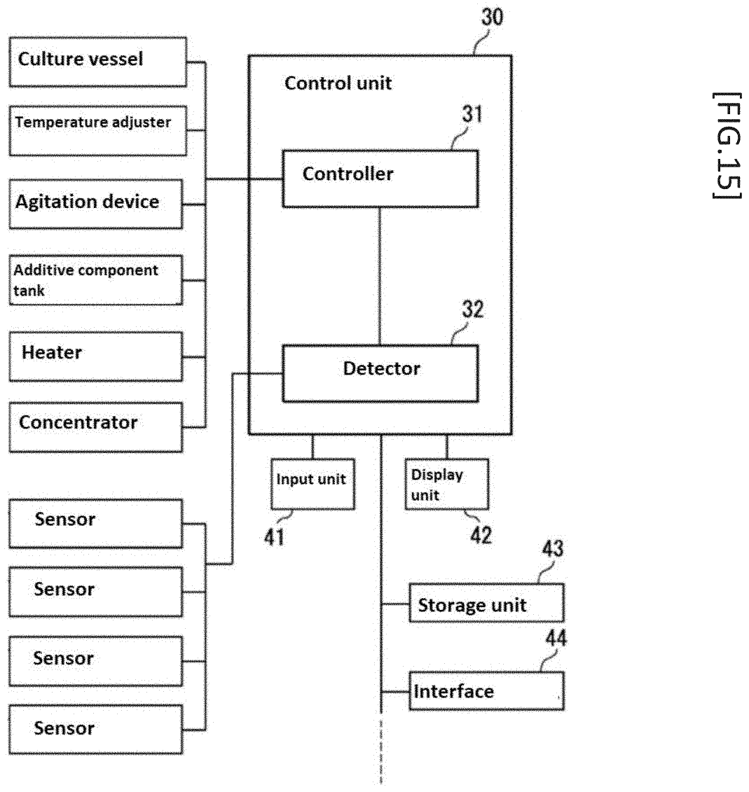

The microalgal product manufacturing system of the present disclosure can have a control unit 30 shown in FIG. 15. The control unit 30 has a controller 31 and a detector 32. The controller 31 and the detector 32 are communicably connected to each other. The system can be configured so that the control described above can be executed with only hardware (e.g., dedicated circuit), or the control described above can be executed by causing a CPU to execute a program.

Data acquired by a sensor (e.g., pH meter, temperature gauge, pressure gauge, oxygen gauge, hardness gauge, ammonium gauge, etc. in a culture vessel) is transmitted to the detector 32, and a signal is transmitted to the controller 31.

The controller 31 is comprised of a CPU (Central Processing Unit), ROM (Read Only Memory), RAM (Random Access Memory), and drive circuits of various actuators included in the microalgal product manufacturing system. In ROM 52, various programs are stored such as BIOS (Basic Input/Output System), OS (Operating System), various drivers, and various applications. The detector 32 is comprised of detection circuits of various sensors (e.g., pH meter and temperature gauge) included in the microalgal product manufacturing system.

The control unit 30 is communicably connected to each of an input unit 41, display unit 42, storage unit 43, and interface 44. The interface 44 allows transmission/reception of data between the control unit 30 and an external apparatus. The control unit 30 is connected to, for example, a generic computer (i.e., personal computer) via the interface 44.

The input unit 41 receives an input from a user. The input unit 41 is comprised of, for example, a keyboard, a mouse, or a touch panel. The display unit 42 is comprised of a display such as an LCD (Liquid Crystal Display) or ELD (Electro Luminescence Display). If the input unit 41 and the display unit 42 are comprised of a touch panel, the input unit 41 and the display unit 42 would be integrated.

The storage unit 43 is comprised of non-volatile memory such as a hard disk. A program associated with various controls, data (e.g., data inputted to the control unit 30 from the input unit 41), and the like are stored in the storage unit 43.

The control unit 31 controls at least one of the constituent elements in a microalgal product manufacturing system such as a culture vessel, a temperature adjustor, an agitation device, an additive component (e.g., nitrogen source, phosphorous source, medium, etc.) tank, a heater, and a concentrator based on at least one of data inputted into the control unit 30 from the input unit 41 and an output signal of a sensor inputted in the detector 32. The length of a liquid feeding tube can be controlled by, for example, switching a flow channel or the like.

(General Technology)

The molecular biological methodologies, biochemical methodologies, and microbiological methodologies used herein are well known or conventional in the art.

(Note)

As used herein, “or” is used when “at least one or more” of the listed matters in the sentence can be employed. When explicitly described herein as “within the range” of “two values”, the range also includes the two values themselves.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. The present disclosure is described hereinafter based on Examples. The above descriptions and the following Examples are not provided to limit the present disclosure, but for the sole purpose of exemplification. Thus, the scope of the present disclosure is not limited to the embodiments and Examples specifically described herein and is limited only by the scope of claims.

### EXAMPLES

The Examples are described hereinafter. For reagents, the specific products described in the Examples were used. However, the reagents can be substituted with an equivalent product from another manufacturer (Sigma-Aldrich, Wako Pure Chemical, Nacalai Tesque, R & D Systems, USCN Life Science Inc., or the like).

### Example 1: Culture of Microalgae

Culture of haptophytes was compared between open culture and photobioreactor culture.

The experiments in this Example used the commercially available Pavlova genus NBRC 102809 strain (Pavlova gyrans), or Pavlova genus OPMS 30543 stain (Pavlova granifera) (accession number NBRC 114066) collected in the sea of Okinawa. The OPMS 30543 strain and NBRC 102809 strain can exhibit the same properties in culture, pheophorbide generation, and fucoxanthin production. A culture media (pH=about 7.5) was prepared by adding a component of Daigo's IMK medium (Nihon Pharmaceutical, Osaka, Japan) to an aqueous solution prepared by dissolving an artificial seawater mix Marine Art SF-1 (Tomita Pharmaceutical, Tokushima, Japan) into water so as to reach 50% seawater concentration, so that the concentration of the component of Daigo's IMK medium would be two fold of the concentration specified in the user manual. While the pH increases with the growth of algal cells, the pH was adjusted to maintain the pH to 8±0.5. The microalgal concentration as of the start of culture was about 0.1 g/L. All culture vessels were installed outdoors and irradiated with only natural light.

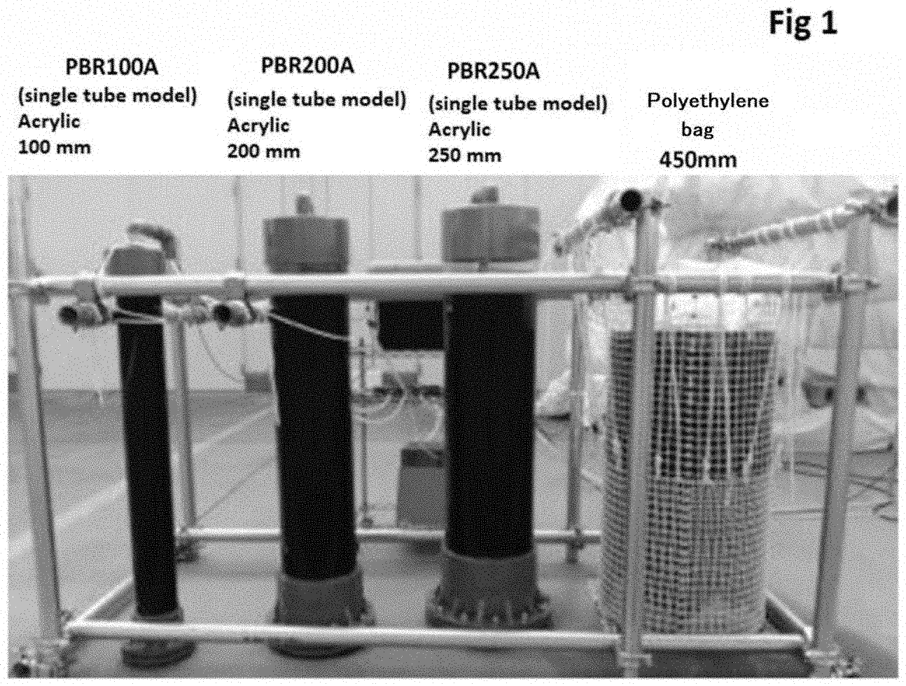

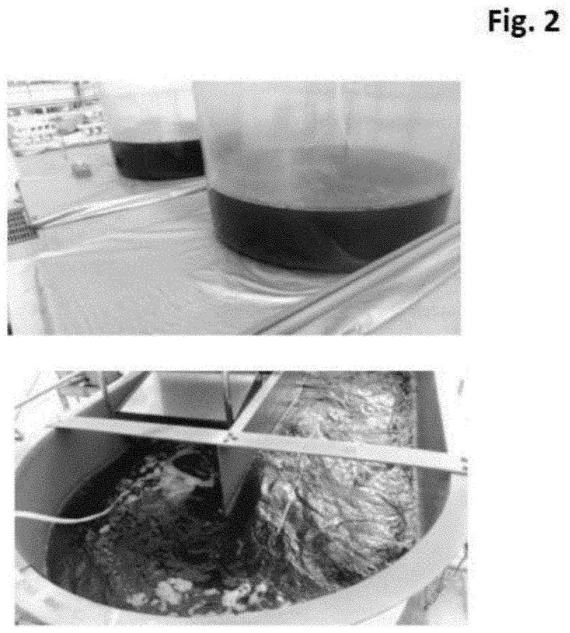

The following culture vessels were used.
photobioreactor (acrylic, 100 mm diameter) (FIG. 1)
photobioreactor (acrylic, 200 mm diameter) (FIG. 1)
photobioreactor (acrylic, 250 mm diameter) (FIG. 1)
photobioreactor (polyethylene bag, 450 mm diameter) (FIG. 1)
500 L tank×2 (FIG. 2)
750 L raceway (FIG. 2)

Only the raceway culture vessel was agitated with a paddle. Other culture vessels were exposed to air for agitation. The 500 L tanks were each cultured with a 200 L medium. The air temperature during the testing period was about 21° C. to about 28° C.

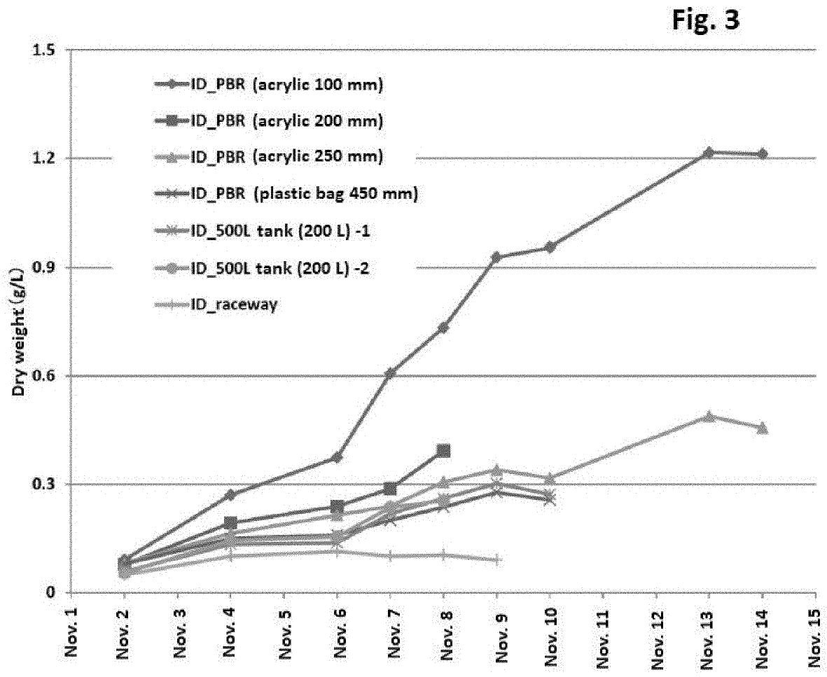

As a result, the results shown in FIG. 3 were obtained. It was found that open culture of haptophytes is difficult, where haptophytes grow to a certain extent, but it is difficult to maintain stable growth due to bacterial contamination. Meanwhile, when a photobioreactor was used, about 12-fold growth was achieved in two weeks, and the cell density reached about 1.2 g/L.

Culture was also attempted using a 1.5 t sheet tank (exposed to air for agitation) under the same conditions described above. After starting culture at a microalgal density of about 0.04 g/L, the microalgal density reached about 0.14 g/L in two weeks, but growth failed due to a subsequent bacterial contamination. Meanwhile, culture was attempted several times with a photobioreactor prepared by the inventors, but hardly any resulted in such a failure.

### Example 2: Design of Photobioreactor

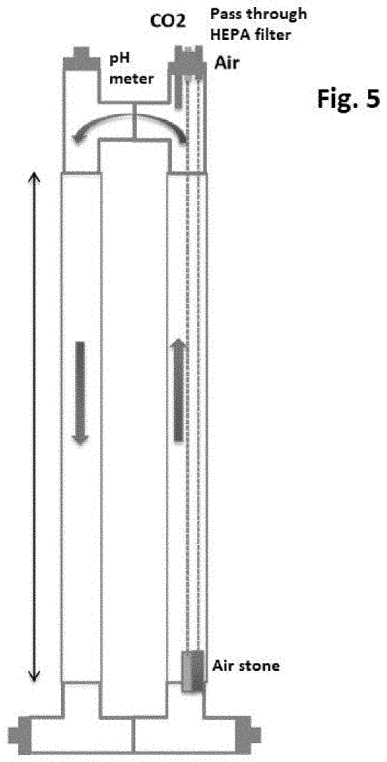

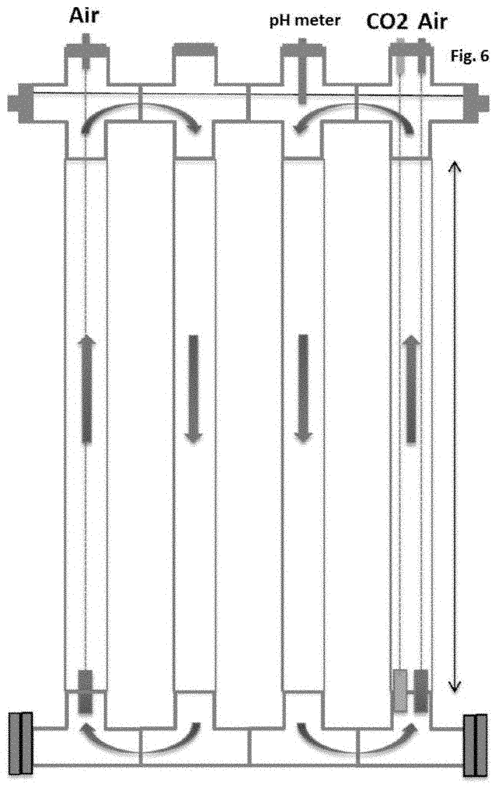

Since it was found that a photobioreactor is suitable for culture of haptophytes, the design of a photobioreactor was optimized (FIGS. 4 and 5). Since the photobioreactor in FIG. 4 uses a thin transparent pipe as a culture vessel, light receiving area is large. Upon exposure to air for agitation, a water flow circulated between the two pipes, thus enabling a more efficient agitation than a photobioreactor with a single pipe. This type of photobioreactor can be further linked to have the configuration shown in FIG. 6. Such a photobioreactor (PBR) has a large light receiving area.

The light receiving areas of each of the culture vessels were compared.

TABLE 1

| | Light receiving area | |
|---|---|---|
| | per 1 L (cm²/L) | |
| 30 L Panlite (water volume: 15 L) | 126 | cm²/L |
| 200 L Panlite (water volume: 150 L) | 63.64 | cm²/L |
| 500 L Panlite (water volume: 200 L) | 44.7 | cm²/L |
| Raceway tank (750 L water) | 32.4 | cm²/L |
| Vinyl Pool (65 L, depth: 10 cm) | 152 | cm²/L |
| Vinyl Pool (130 L, depth: 20 cm) | 102 | cm²/L |
| Sheet vessel 1.5 ton (3.6 m × 1.8 m × 0.25 m) | 43.2 | cm²/L |
| PBR 50A (two tube model) | 738.5 | cm²/L |
| PBR 100A (two tube model) | 233.9 | cm²/L |
| PBR 100A (single tube model) | 380.4 | cm²/L |
| PBR 200A (single tube model) | 194.05 | cm²/L |
| PBR 250A (single tube model) | 155.5 | cm²/L |
| Open pond (ϕ 3 m * 15 cm depth) | 70.65 | cm²/L |

The pH was adjusted to 8 by adding CO₂ to an IMK×2 medium to which artificial seawater was added so as to reach 50% seawater concentration in the same manner as Example 1. About 0.1 g/L of the Pavlova strains described above was added to the medium. The microalgae were cultured while being exposed to air for agitation in the photobioreactor in FIG. 4 outdoors under natural light. The air temperature during the testing period was about 21° C. to about 28° C.

The results are shown in FIG. 5. In about one week, about a 10-fold growth was achieved, and the cell density reached about 1.1 g/L.

Long-term culture was performed using the same culture vessel (FIG. 6). As a result, stable continuous culture was achieved over about 40 days. Despite recovering algal bodies several times, a high cell density of about 3.5 g/L was able to be maintained.

Since bacterial contamination is reduced in microalgae cultured in a photobioreactor described above, it is expected that microalgae can be recovered after sufficient growth, prior to an incident of bacterial contamination, even in open culture by performing seed culture in such a photobioreactor and then performing open culture.

### Example 3: Recovery of Microalgae

Haptophytes have a feature of being relatively soft due to the lack of cell walls. Haptophytes are also relatively small microalgae that are about 1 to 10 μm. A method that can efficiently recover such soft and small haptophytes was studied.

The Pavlova strains described above (0.516 g/L) were centrifuged or filtered (MF membrane) to concentrate the algal body 100-fold. After concentration, the state of cells was observed under a microscope.

HITACHI himac CR22GII (Hitachi, Ltd., Tokyo, Japan) was used for centrifugation. Centrifugation was performed for about 10 minutes at 5000 rpm. Since approximately 3 L can be concentrated with a single run of centrifugation, this was repeated about three times for centrifugation of 10 L. The concentrates were centrifuged about one or two more times (about 30 to 60 minutes) to consolidate the concentrates.

For filtering, a Microza AHP1010D (Asahi Kasei, Tokyo, Japan) (ultrafilter, molecular weight cut-off of 50 KDa, cross-flow filtration) membrane and a magnet pump MD-15RV-N (Iwaki, Tokyo, Japan) (discharge rate: 16/19 L/min) were used to produce a filtrate at a rate of 50 to 100 mL/min. The concentration period was about 6 L/hr.

It was confirmed that haptophytes can be recovered without destroying cells by both centrifugation and filtering methods.

### Example 4: Generation of Pheophorbide in Microalgae

The inventors performed component analysis on the Pavlova stains described above. It was found that the strains comprise about 2250 mg/100 g (dry weight) chlorophyll when measured by absorptiometry (visible).

As a result, Pavlova, which is haptophyte, was found to contain a greater amount of chlorophylls relative to common microalgae such as Chlorella.

It is known that chlorophylls are converted to pheophorbide by being metabolized in microalgae. It is also known that pheophorbide induces photosensitivity or the like, and it is desirable that intake by animals is restricted. The inventors studied whether pheophorbide is generated during culture and recovery of Pavlova.

10 L of culture of the Pavlova strains described above (0.516 g/L) was centrifuged and concentrated 100-fold. The concentrate was then heated by autoclave (100° C., 1 minute). The existing pheophorbide and chlorophyllase activity level were each measured as follows. The amount of total pheophorbide=amount of existing pheophorbide+chlorophyllase activity level.

### Quantification Method of Existing Pheophorbide

The amount of chlorophyll decomposition products migrating from an ether extraction solution of pigments to 17% hydrochloric acid is converted to pheophorbide a (mg %).

100 mg of dried microalgae is measured out into a mortar. About 0.5 g of sea sand and 20 ml of 85% (V/V) acetone are added. The microalgae are quickly ground and then the supernatant is transferred to a centrifuge tube. 10 ml of acetone is further added to the residue to perform the same process, and the supernatant is transferred to a centrifugation tube. This process is repeated one more time. Then following a centrifugation (3000 rpm, 5 minutes), the supernatant is transferred to a separatory funnel containing 30 ml of ethyl ether. 50 ml of 5% sodium sulfate solution is then added to the ether-acetone mixture. The resulting mixture is gently shaken, and the sodium sulfate layer is discarded. After repeating this washing process three times, anhydrous sodium sulfate is added for dehydration. The ether layer is retrieved, and the total amount is adjusted to 50 ml with ethyl ether as the stock pigment solution. 20 ml of the stock pigment solution is sequentially subjected to shaking extraction with 20 ml of 17% hydrochloric acid and then 10 ml of the same hydrochloric acid, and then the hydrochloric acid layer is transferred to a separatory funnel containing 150 ml of saturated sodium sulfate solution and 20 ml of ethyl ether. This is subjected to shaking extraction. The ether layer is fractionated. Ethyl ether is added thereto to adjust the total amount to 20 ml as the decomposition product extract. The decomposition product extract is diluted with ethyl ether until precisely reaching the required concentration, and the absorbance at 667 nm is measured. The amount of chlorophyll decomposition product is calculated from the absorbance of the reference standard pheophorbide a as the amount of existing pheophorbide (mg %). For the absorbance of the reference standard pheophorbide a, the specific absorbance coefficient 70.2 (absorbance indicated by 0.1% solution, 1 cm) at 667 nm of pheophorbide a of S. R. Brown (J. Fish Res. Bd. Canada 25, 523-540. 1968) was used.

Quantification Method of Chlorophyllase Activity Level

Microalgae are incubated in water-containing acetone, and the amount of increase in the generation of chlorophyll decomposition product is converted into the amount of pheophorbide a (mg %).

100 mg of dried microalgae is precisely weighted out. 10 mL of a mixture of cooled M/15 phosphate buffer (pH 8.0) and acetone (7:3) is added thereto. The resulting mixture is incubated for 3 hours at 37° C. The mixture is then adjusted to be weakly acidic with 10% hydrochloric acid. The amount of pheophorbide is measured by the same method as the quantification method of existing pheophorbide. The amount of existing pheophorbide is subtracted from the measurement value to find the amount of increase. The amount of increase is used as the chlorophyllase activity level.

TABLE 2

| | Stock | 100-fold |
|---|---|---|
| | solution | concentrate |
| Amount of existing pheophorbide (mg %) | 17.91 | 150.72 |
| Amount of total pheophorbide (mg %) | 20.39 | 135.51 |
| Chlorophyllase activity level (mg %) | 2.48 | −15.21 |

Both existing pheophorbide and total pheophorbide were low in unconcentrated culture, but the amount of existing pheophorbide increased when centrifuged. Since chlorophyllase activity was suppressed by heating, the amount of total pheophorbide was comparable to the amount of existing pheophorbide.

Since the amount of existing pheophorbide in 100-fold concentrate was about 9-fold as compared to the stock solution, the amount of stress from the concentration process described above is predicted to be about 9.

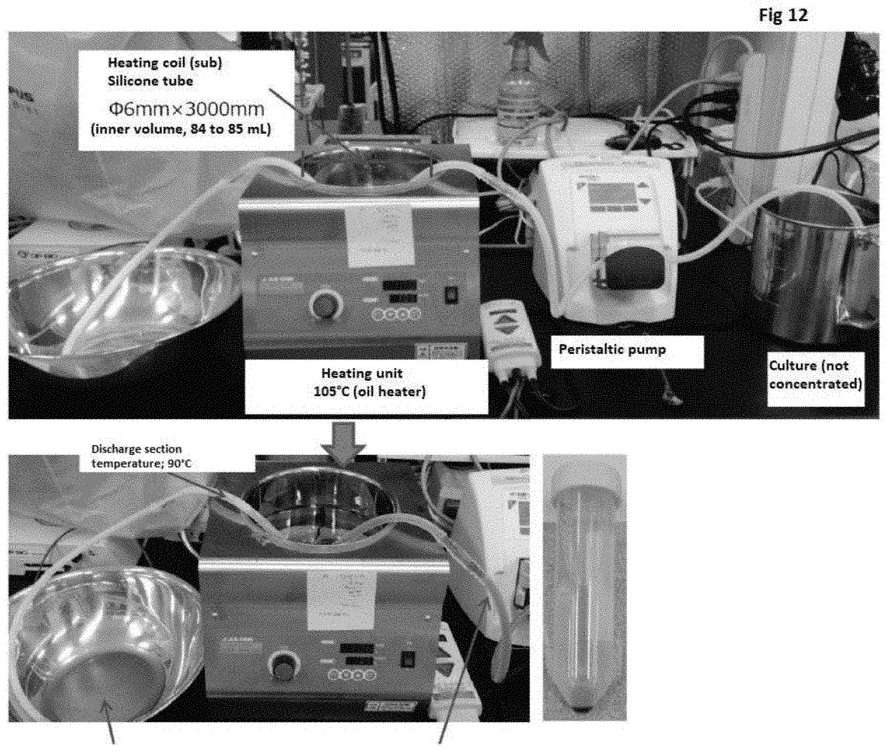

10 L of culture of the Pavlova strains described above (0.516 g/L) was concentrated 100-fold with an MF membrane in the same manner as the concentration by filtering in Example 3 (about 12 hours or more). The concentrate was then heated (2 minutes or 4 minutes at 110° C.) with a coil heater (FIG. 12). An increase in the amount of existing pheophorbide was observed for each sample when measured in the same manner as the method described above. While the damage to cells was milder with concentration using an MF membrane as compared to centrifugation in view of microscopic observation, it was found that the amount of stress was in fact significant in view of the increase in the amount of existing pheophorbide. Since the amount of existing pheophorbide was also high in samples subjected to the heating described above, it is possible that the amount of existing pheophorbide cannot be sufficiently suppressed when cells are heated at a high cell density.

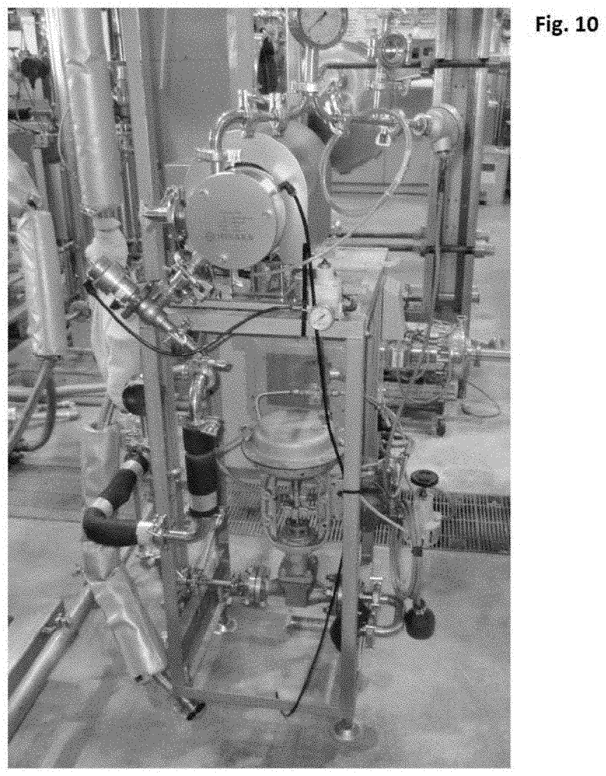

Furthermore, increase in pheophorbide production due to model stimulation was tested. Parts of a culture retrieved after passing culture L of the Pavlova strains described above (1.482 g/L) through a cascade pump (FIG. 10) were evaluated as samples. Samples subjected to 0 passes through the pump, 1 pass through the pump, 2 passes through the pump (90 seconds), 3 passes through the pump (135 seconds), 5 passes through the pump (225 seconds), 10 minute circulation through the pump, and 20 minute circulation through the pump were evaluated. After microscope observation of each sample, the samples were subjected to coil heating (110° C., 4 minutes), centrifugation, and lyophilization. The existing pheophorbide, total pheophorbide, and chlorophyllase activity level were measured in the same manner as the method described above for the dried products thereof (10 mg).

TABLE 3

| | 0 passes | 1 pass | 2 passes | 3 passes | More (2) |
|---|---|---|---|---|---|
| | 0 sec | 45 sec | 90 sec | 135 sec | 20 min |
| Amount of | 36.91 | 53.59 | 47.48 | 70.54 | 83.67 |
| existing | | | | | |
| pheophorbide | | | | | |
| (mg %) | | | | | |
| Amount of total | 64.31 | 55.16 | 64.69 | 69.38 | 58.09 |
| pheophorbide | | | | | |
| (mg %) | | | | | |
| Chlorophyllase | 27.40 | 1.58 | 17.21 | −1.16 | −25.59 |
| activity level | | | | | |
| (mg %) | | | | | |

The amount of existing pheophorbide increased more with greater increase in physical impact. It is conjectured that in addition to physical impact, increase in the temperature of the culture after passage also contributed to the increase in the amount of existing pheophorbide.

The amounts of existing pheophorbide were about 1.4-fold, about 1.3-fold, about 1.9 fold, and about 2.3-fold for 1 pass through the pump, 2 passes through the pump, 3 passes through the pump, and 20 minute circulation through the pump, respectively, with respect to 0 passes through the pump. Thus, the amount of stress under each of the shearing loads described above is predicted to be about 1.3 to 2.3.

It was found from these results that the amount of pheophorbide can increase through manipulation of microalgae. For this reason, a method for reducing pheophorbide was examined.

### Example 5: Suppression of Pheophorbide by Heating

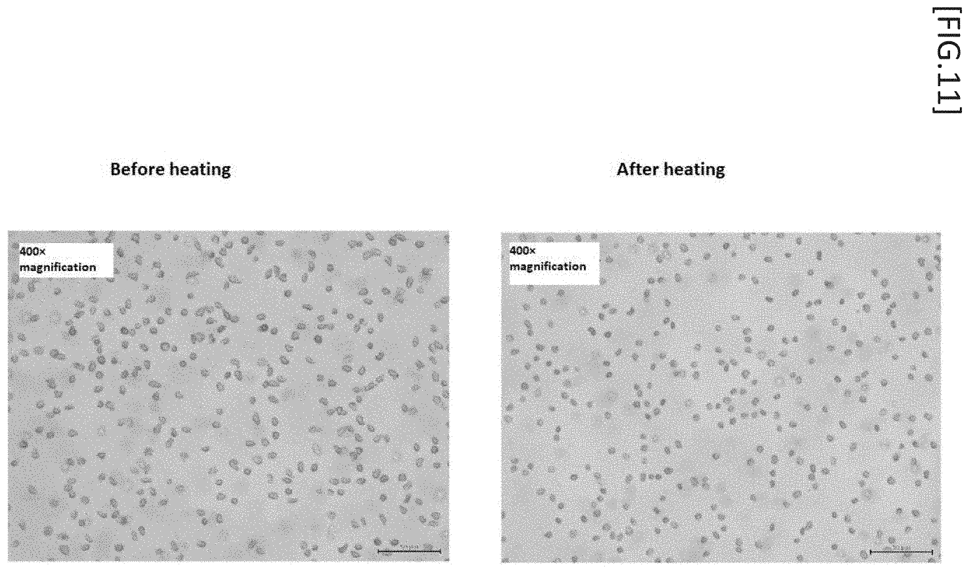

When the Pavlova strains described above were heated, the color of the algal body, which was close to brown, changed to vivid green. Rupture in the algal body was not observed (FIG. 11). Since it was assumed that chlorophyllases, which catalyze the production of pheophorbide, are inactivated by heating, a test was conducted to determine whether pheophorbide production is suppressed by heating.

60 L culture (0.145 g/L) was concentrated to 0.6 L over about 10 hours at 28° C. by the same filtering process as Example 4 (100-fold concentration: 13.440 g/L). Cells were observed under a microscope after concentration, but an abnormality was not observed. Some of the concentrate was heated for 4 minutes at 95° C. or higher with the apparatus shown in FIG. 12.

When pheophorbide was examined, the amount of existing pheophorbide was increased in heated concentrates as compared to non-heated concentrates. It is understood that this is due to insufficient inactivation of chlorophyllase. Chlorophyllase inactivation was insufficient, possibly due to: thermal conductivity of a solution decreased in view of the high solid density in the solution (1% to 1.5%) such that the cells at the inner part could not be sufficiently heated at the specified temperature; increased thermal insulation due to increase in the density of extracellular substances (protein, polysaccharide, etc.); and the like.

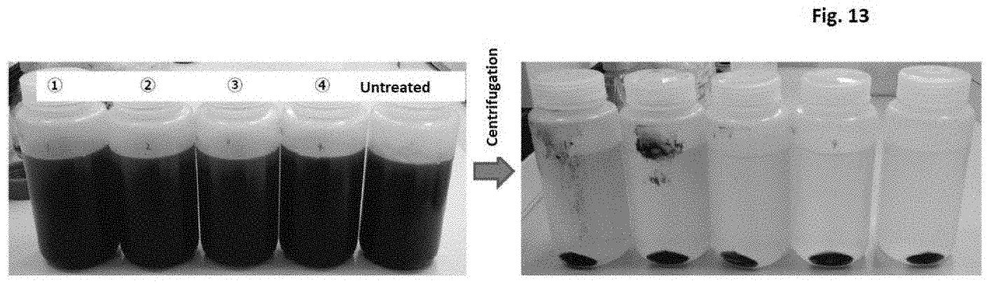

A heating apparatus was configured as shown in FIG. 12. A culture (0.592 g/L) of the Pavlova strains described above was fed to an oil heater (105° C.) at a certain rate (10, 20, 40, or 80 mL/minute) through a tube to adjust the heating time. The heated solution from the oil heater was collected in a bottle on ice. The heating times under each condition were about 8 minutes, about 4 minutes, about 2 minutes, and about 1 minute. Each of the recovered samples was centrifuged (FIG. 13). The existing pheophorbide, total pheophorbide, and chlorophyllase activity level were measured in the same manner as the method described above.

TABLE 4

| | (1) Coil | (2) Coil | (3) Coil | (4) Coil | |
|---|---|---|---|---|---|
| | (heating, | (heating, | (heating, | (heating, | (5) No |
| | 8 minutes) | 4 minutes) | 2 minutes) | 1minute) | heating |
| Amount of | 37.18 | 35.89 | 70.10 | 155.36 | 41.40 |
| existing | | | | | |
| pheophorbide | | | | | |
| (mg %) | | | | | |
| Amount of | 38.30 | 39.00 | 40.60 | 1135.13 | 801.53 |
| total | | | | | |
| pheophorbide | | | | | |
| (mg %) | | | | | |
| Chlorophyllase | 1.12 | 3.11 | −29.49 | 979.77 | 760.14 |
| activity level | | | | | |
| (mg %) | | | | | |

It was found that with sufficient heating, the amount of total pheophorbide does not increase, even when centrifuged thereafter. The amount of existing pheophorbide increased more in a sample heated for 1 minute than a sample that was not heated, but it is understood that this is because inactivation of chlorophyllase was insufficient, and chlorophyllases released in view of cell disruption due to heating acted on a broad range of chlorophylls.

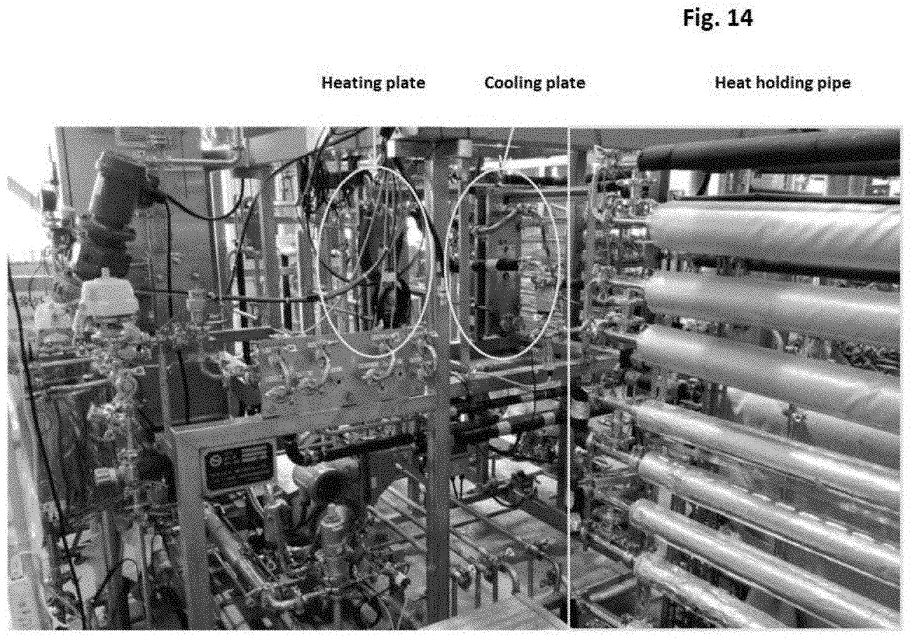

Furthermore, the effect of suppressing pheophorbide by plate heating shown in FIG. 14 was tested.

TABLE 5

| | 100° C., | No | 110° C., | 100° C., |
|---|---|---|---|---|
| | 4 minutes | heating | 4 minutes | 4 minutes |
| | (coil) | (reference) | (plate) | (plate) |
| Amount of | 63.97 | 35.93 | 57.10 | 61.15 |
| existing | | | | |
| pheophorbide | | | | |
| (mg %) | | | | |
| Amount of total | 84.35 | 1079.13 | 76.21 | 86.29 |
| pheophorbide | | | | |
| (mg %) | | | | |
| Chlorophyllase | 20.37 | 1043.19 | 19.11 | 25.15 |
| activity level | | | | |
| (mg %) | | | | |

It was found that pheophorbide production is also suppressed by plate heating.

In view of the above results, the amount of pheophorbide was successfully suppressed by suppressing chlorophyllase activity while controlling the amount of stress.

### Example 6: Stability of Fucoxanthin During Treatment of Microalgae

The inventors found that haptophytes such as the Pavlova strains described above are rich in fucoxanthin. While fucoxanthin is a useful component, it is known as a substance that is chemically unstable and readily decomposed by heat or the like. A test was conducted to determine whether fucoxanthin in haptophytes decomposes during heating.

Fucoxanthin was quantified by comparing a sample with a reference standard fucoxanthin (99%) purchased from Wako Pure Chemical (Tokyo, Japan) through HPLC analysis. The following are the conditions for the HPLC analysis.
Column: Cosmosil 5C18AR-II, inner diameter 4.6×100 mm
Column temperature: 40° C.
Solvent: aqueous 72.5% acetonitrile solution (0.1% formic acid), 20 minute elusion
Flow rate: 1 mL/min
Detection: 450 nm wavelength
Amount introduced: 20 μL

Decomposition of fucoxanthin by heating a moist sample immediately after culture was studied.

A culture of the Pavlova strains described above was filtered with a filter to obtain filtered algal bodies. The filtered algal bodies were dried under the condition of lyophilization, 60° C. for 1 hour, or 75° C. for 30 minutes. Water and acetonitrile were added to the dried samples, and fucoxanthin was extracted. The extract was transferred to a tube and centrifuged (12000 rpm, 3 min). The supernatant was analyzed by HPLC. As a result, the amounts of fucoxanthin shown in the following table were determined.

TABLE 6

| Condition | Mean fucoxanthin amount at n = 3 (mg/g) |
|---|---|
| Lyophilization (no heating) | 6.13 |
| 60° C., 1 hour | 4.87 |
| 75° C., 30 minutes | 4.82 |

Decomposition of fucoxanthin was observed under heating conditions of 60° C. for 1 hour and 75° C. for 30 minutes.

Decomposition of fucoxanthin by heating lyophilized samples was studied.

Lyophilized samples were prepared. The lyophilized samples were processed under the condition of no heating, 120° C. for 1 hour, or 170° C. for 30 minutes.

Water and acetonitrile were added to the heated samples, and fucoxanthin was extracted. The extract was transferred to a tube and centrifuged (12000 rpm, 3 min). The supernatant was analyzed by HPLC. As a result, the amounts of fucoxanthin shown in the following table were determined.

TABLE 7

| Condition | Mean fucoxanthin amount at n = 3 (mg/g) |
|---|---|
| Lyophilization (no heating) | 9.67 |
| 120° C., 1 hour | 6.5 |
| 170° C., 30 minutes | 0.09 |

Decomposition of fucoxanthin was observed under heating conditions of 60° C. for 1 hour and 75° C. for 30 minutes.

Decomposition of fucoxanthin by coil heating (FIG. 12) in Example 5 was studied, yielding the results in the following table.

TABLE 8

| | Amount of fucoxanthin (mg/g) |
|---|---|
| (1) heating, 8 minutes | 11.00 |
| (2) heating, 4 minutes | 11.91 |
| (3) heating, 2 minutes | 12.46 |
| (4) heating, 1 minute | 11.71 |
| (5) no heating | 10.19 |

It is predicted that the amount of fucoxanthin increased in heated samples because the cells shrunk due to heating, thus reducing the unit weight of algal body.

A decrease in the amount of fucoxanthin was not observed even under the coil heating condition for suppressing pheophorbide production studied in Example 5.

Decomposition of fucoxanthin by plate heating in Example 5 was studied, yielding the results in the following table.

TABLE 9

| | 110° C., | Not | 110° C., | 100° C., |
|---|---|---|---|---|
| | 4 minutes | heated | 4 minutes | 4 minutes |
| | (coil) | (reference) | (plate) | (plate) |
| Amount of existing | 63.97 | 35.93 | 57.10 | 61.15 |
| pheophorbide (mg %) | | | | |
| Amount of total | 84.35 | 1079.13 | 76.21 | 86.29 |
| pheophorbide (mg %) | | | | |
| Chlorophyllase activity | 20.37 | 1043.19 | 19.11 | 25.15 |
| level (mg %) | | | | |
| Amount of Fx (mg/g) | 15.93 | 14.31 | 12.33 | 13.84 |
| Ratio of Fx (%) | 111.3 | 100 | 86.2 | 96.7 |

A decrease in the amount of fucoxanthin was not observed even under the plate heating condition for suppressing pheophorbide production studied in Example 5.

### Example 6X: Treating Other Microalgal Species

The pheophorbide generation suppressing conditions and fucoxanthin decomposition reducing conditions are also examined for microalgae of the Isochrysis genus (I. galbana, I. litoralis, I. maritima, Tisochrysis lutea, etc.) in the same manner as the method described above. The amount of pheophorbide generation when stress (concentration, shearing, etc.) is applied to microalgae of the Isochrysis genus is studied. Chlorophyllase inactivation treatment (e.g., heating) is performed under conditions that do not apply stress, or apply moderate stress (concentrating by 1.5-fold, 2-fold, or the like), to microalgae of the Isochrysis genus to study the suppression of pheophorbide generation. The levels of fucoxanthin decomposition under the conditions described above are also studied. The scope of treatment conditions with low fucoxanthin decomposition without excessive pheophorbide generation is determined from the results.

### Example 7: Microalgal Product

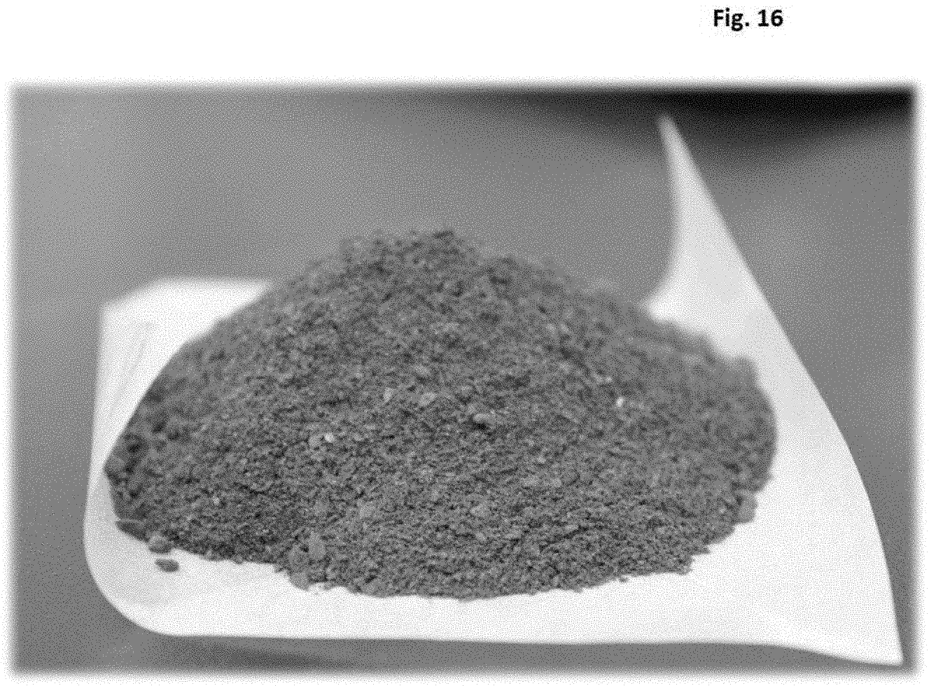

The strains described above were dried and powderized to prepare a food product (FIG. 16). The outer appearance has a natural green color. The product had an iwanori-like flavor. It was confirmed that Pavlova can be suitably used as food products.

### Example 8: Oil Immersed Product

The preservability of algal body manufactured above was tested. A culture of the strains described above was heated for 4 minutes at or above 100° C. in the same manner as the Examples described above, then centrifuged to prepare a Pavlova concentrate. To the concentrate, tap water was added at about 10 times the amount of the concentrate. The mixture was agitated for about 3 to about 4 hours, and then centrifuged again for desalination. The desalinated algal bodies were frozen. Dried algal bodies were then prepared in accordance with the following procedure.
Frozen algal bodies were thawed under conditions of room temperature, flowing water, and warm bath, which were mostly heating condition of at most about 60° C.
The algal bodies were sterilized for about 1 minute to 1 hour at about 80 to 85° C.
Foreign objects were removed with a strainer and a magnet with a magnetic flux density of 10000 G.
The algal bodies were pre-frozen at −18° C. to −60° C.
The algal bodies were lyophilized for 48 hours or longer. The inside of a commercially available lyophilizer was pre-cooled to −20° C. to −80° C. The pre-frozen algal bodies were placed in a manifold or a chamber in accordance with the amount and usage. The pressure inside the lyophilizer was reduced to 20 Pa or less with a vacuum pump, and the algal bodies were lyophilized for 24 to 48 hours or more in accordance with the amount used. In some cases, the temperature within the chamber was heated to 10° C. to 20° C.
The lyophilized product was pulverized to the extent that the cells would not be crushed using a dispensing spoon or mortar. For particularly large amounts, a pulverizer was used.
Foreign objects were removed with an 80 mesh sieve and a magnet with a magnetic flux density of 12000 G.

### Testing Conditions

### (Dry Product)

250 mg or more dry algal bodies were placed in a vinyl packaging for each measurement point (test samples: n=3).
A desiccant and deoxygenation agent were added to the vinyl packaging as needed, and the vinyl packaging was placed in an aluminum packaging. Desiccant: silica gel (FUJIGEL SANGYO, Osaka, Japan); deoxygenation agent, Vitalon (Tokiwa Sangyo, Kanagawa, Japan).
Algal bodies were left standing away from light under respective set temperature conditions (refrigeration; 5° C., normal temperature; 20° C. to 28° C., high temperature; 40° C.)
The remainder as of the preparation of the sample was analyzed in the measurement upon starting.
A small amount was recovered and analyzed from each sample at each point thereafter (after 2 weeks, after 1 month, each month after 2 to 12 months, after 15 months, after 18 months, after 21 months, and after 24 months).
The analysis measured the amount of fucoxanthin and pheophorbide (used mixture of n=3 samples).

### (Oil Immersed Product)

12 g of dry algal bodies were mixed and kneaded with 12 mL of oil or 12 mL of vitamin E-added oil. Oil, olive oil (Fujifilm Wako Pure Chemical, Osaka, Japan); a tocopherol, (Fujifilm Wako Pure Chemical, Osaka, Japan).
After confirming that oil content and algal bodies were mixed well, about 100 mg of the oil-algal body mixture was measured out and placed in an Eppendorf tube (made of polypropylene, 2 mL capacity). (Test samples: n=3).
The samples were left standing away from light under respective set temperature conditions (refrigeration; 5° C., normal temperature; 20° C. to 28° C., high temperature; 40° C.)
The remaining oil-algal body mixture as of the preparation of the sample was analyzed in the measurement upon starting.
A small amount was recovered and analyzed from each sample at each point thereafter (after 2 weeks, after 1 month, and once monthly thereafter).
The analysis measured the amount of fucoxanthin.

Fucoxanthin was extracted in the following manner, and fucoxanthin was measured in the same manner as Example 7.
100% ethanol is added to samples at each point for extraction with ultrasonication for 10 minutes.
The mixture is centrifuged (12000 rpm, 2 minutes) to separate the mixture into extract and algal bodies.
Only the required amount of extract is recovered for measurement by HPLC.

The results of fucoxanthin measurement are shown below.

TABLE 10
Fucoxanthin in dry product (ratio with respect to amount as of start (%))

| | | | Deoxygen- | | | | | | | | | | |
|---|---|---|---|---|---|---|---|---|---|---|---|---|---|
| | | | eation | | | | | | | | | | |
| | Temperature | Desicant | agent | 2 W | 1 M | 2 M | 3 M | 4 M | 5 M | 6 M | 7 M | 8 M | 9 M |
| A | Refrigerated | — | — | 100 | 99 | 88 | 92 | 80 | 72 | 64 | 57 | 42 | 32 |
| B | Normal temp | — | — | 51 | 59 | 39 | 33 | 29 | 21 | 17 | 11 | 8 | 4 |
| C | Normal temp | ◯ | — | 91 | 76 | 52 | 48 | 38 | 37 | 6 | 30 | 27 | 27 |
| D | Normal temp | ◯ | ◯ | 102 | 99 | 89 | 88 | 86 | 77 | 76 | 71 | 68 | 66 |
| E | High temp | — | — | 34 | 15 | 4 | 3 | 2 | 1 | 2 | — | — | — |
| F | High temp | ◯ | — | 32 | 13 | 4 | 6 | 4 | 3 | 3 | — | — | — |
| G | High temp | ◯ | ◯ | 51 | 35 | 21 | 10 | 4 | 1 | 0 | — | — | — |

TABLE 11
Fucoxanthin in oil immersed product (ratio with respect to
amount as of start (%))

| | Temperature | Vitamin E | 2 W | 1 M | 2 M | 3 M | 4 M | 5 M | 6 M | 7 M | 8 M | 9 M |
|---|---|---|---|---|---|---|---|---|---|---|---|---|
| A | Refrigerated | — | 100 | 101 | 91 | 95 | 98 | 92 | 89 | 91 | 85 | 86 |
| B | Refrigerated | ◯ | 100 | 94 | 90 | 94 | 93 | 94 | 94 | 88 | 85 | 84 |
| C | Normal temp | — | 96 | 94 | 97 | 82 | 78 | 72 | 65 | 57 | 44 | 44 |
| D | Normal temp | ◯ | 84 | 79 | 73 | 72 | 72 | 76 | 67 | 63 | 59 | 50 |
| E | High temp | — | 55 | 28 | 8 | 5 | 4 | 3 | 2 | — | — | — |
| F | High temp | ◯ | 51 | 31 | 16 | 10 | 7 | 4 | 5 | — | — | — |

It was found that fucoxanthin can be maintained well even at normal temperatures in a dry product by adding a desiccant+deoxygenation agent. It was found that fucoxanthin can be maintained well even at normal temperatures in an oil immersed product, and stability can be further improved by adding vitamin E. It was speculated that reduction in fucoxanthin is sufficiently suppressed, especially under conditions of storage by freezing at or below −20° C., storage of a dry product added with a desiccant+deoxygenation agent at or below 5° C., or storage by immersion in oil at or below 5° C.

The results of measuring pheophorbide in a dry product are shown below.

TABLE 12
Amount of existing pheophorbide in dry product (mg %)

| | | | Deoxygen- | | | | | | | | | | |
|---|---|---|---|---|---|---|---|---|---|---|---|---|---|
| | | | cation | | | | | | | | | | |
| | Temperature | Desicant | agent | 2 W | 1 M | 2 M | 3 M | 4 M | 5 M | 6 M | 7 M | 8 M | 9 M |
| A | Refrigerated | — | — | 42 | 57 | 51 | 60 | 51 | 51 | 53 | 47 | 33 | 53 |
| B | Normal temp | — | — | 49 | 61 | 53 | 47 | 46 | 36 | 38 | 23 | 19 | 27 |
| C | Normal temp | ◯ | — | 41 | 71 | 64 | 60 | 61 | 56 | 37 | 50 | 36 | 64 |
| D | Normal temp | ◯ | ◯ | 59 | 71 | 60 | 63 | 86 | 59 | 73 | 70 | 45 | 81 |
| E | High temp | — | — | 58 | 46 | 27 | 18 | 19 | 14 | 12 | — | — | — |
| F | High temp | ◯ | — | 43 | 53 | 33 | 94 | 43 | 38 | 42 | — | — | — |
| G | High temp | ◯ | ◯ | 54 | 112 | 65 | 89 | 89 | 83 | 76 | — | — | — |

An increase in the amount of existing pheophorbide was not observed under any condition, regardless of temperature. It is expected that storage in a dry state does not result in significant generation of pheophorbide.

### Example 9: Frozen Product

The microalgae described above are frozen by the following procedure.
A microalgal concentrate prepared in the same manner as the Examples described above is frozen (at or below −40° C.), or enclosed (preferably sealed/vacuum packed) in a nylon bag or aluminum bag that can withstand sterilization by boiling (80 to 100° C.) or retort sterilization, and quickly frozen at or below −40° C. Upon enclosure, the concentrate is degassed and infused and/or a frozen item is vacuum packed as needed.
An excipient (cyclodextrin, etc.), antioxidant, emulsifier, and/or thickener is added as needed.
Fruit juice, fruit extract, and/or flavoring is added as needed. The flavor of microalgae can be masked.
A dairy product is added as needed to prepare a LACT-ICE, an ICE-MILK, or an ICE-CREAM.
The frozen product is shaped in a sheet-like shape or frozen in a sheet-like form.

(Notes)

As disclosed above, the present disclosure is exemplified by the use of its preferred embodiments. However, it is understood that the scope of the present invention should be interpreted based solely on the Claims. It is understood that any patent, any patent application, and any references cited herein should be incorporated herein by reference in the same manner as the contents are specifically described herein.

### INDUSTRIAL APPLICABILITY

The present disclosure provides a safe microalgal product with reduced pheophorbide, and a manufacturing method and system that enable efficient provision thereof. Such microalgal products can provide various health, nutritional, and/or cosmetic effects. Further, a high quality microalgal product can be provided with low environmental impact by using such a manufacturing method and system. The present disclosure also provides a culturing apparatus that enables high concentration culture with little bacterial contamination, which enables highly convenient culture of microalgae.

1. A method for manufacturing a microalgal product, comprising:
(A) a step of maintaining microalgae under a condition where an amount of stress applied to the microalgae from after culture to step (B) is controlled to a predetermined value or less, wherein the step maintains a density of the microalgae at or below a predetermined value and/or does not concentrate the microalgae by a predetermined factor or greater; and
(B) a step of subjecting the microalgae to treatment for inactivating a chlorophyllase, and
wherein the microalgae are microalgae of the class Haptophyceae.

2. The method of claim 1, wherein the predetermined value of the density and/or the predetermined factor of the concentration is determined based on an increase in pheophorbide upon concentrating the microalgae.

3. The method of claim 1, wherein the predetermined value of the density is less than or equal to about 10 g/L (dry weight).

4. (canceled)

5. The method of claim 1, wherein the predetermined factor of the concentration is greater than or equal to about 100-fold.

6. (canceled)

7. The method of claim 1, wherein the predetermined value of the amount of stress is less than or equal to about 5.

8. (canceled)

9. (canceled)

10. The method of claim 1, wherein treatment for concentrating the microalgae is not performed in step (A).

11. The method of claim 1, wherein a step of culturing the microalgae comprises growing the microalgae to a density greater than or equal to 1 g/L (dry weight).

12. The method of claim 1, comprising a step of concentrating the microalgae after step (B).

13. The method of claim 1, wherein step (B) comprises heating the microalgae.

14. (canceled)

15. The method of claim 1, wherein step (B) is performed under a condition that does not decompose fucoxanthin, or where a reduction in fucoxanthin after step (B) in comparison to before the step is less than 80%.

16. (canceled)

17. The method of claim 1, comprising a step of drying the microalgae after step (B).

18. The method of claim 1, wherein the microalgae produce 30 mg or more chlorophyll per 1 g of dry weight.

19. The method of claim 1, wherein the microalgae are algae that produce fucoxanthin.

20. The method of claim 1, wherein the microalgae are algae that produce 8 mg or more fucoxanthin per 1 g of dry weight.

21.-61. (canceled)

62. The method of claim 1, wherein the microalgae are microalgae of the family Pavlovaceae.

63. The method of claim 1, wherein the microalgae are microalgae of the genus Pavlova.

64. The method of claim 1, wherein the microalgae are P. calceolate, P. granifera, P. gyrans, P. lutheri, P. pinguis, or P. salina.

65. The method of claim 20, wherein the microalgae are P. granifera or P. gyrans.

66. A microalgal product comprising an algal body of the microalgae of the class Haptophyceae.

67. The microalgal product of claim 66, wherein pheophorbide content of the microalgae is less than or equal to 0.2% by weight.

68. The microalgal product of claim 66, wherein the microalgae are P. granifera or P. gyrans.

69. A method of using the microalgal product of claim 66 as an edible product, a food product or a cosmetic product.

70. The microalgal product of claim 66, wherein fucoxanthin content is greater than or equal to 0.8% by weight.

71. The microalgal product of claim 66, wherein fucoxanthin content of the microalgae is greater than or equal to 0.8% by weight (dry weight).

72. The microalgal product of claim 66, wherein chlorophyll content of the microalgae is greater than or equal to 3% by weight (dry weight).

73. A method for manufacturing a frozen product, comprising:
a step of preparing a microalgal concentrate by the method of claim 1; and
a step of freezing the microalgal concentrate.

74. The method of claim 73, wherein the freezing step comprises cooling to −40° C. or lower.

75. The microalgal product of claim 66, which is dried product, a tablet, powder, capsule, cream, a frozen product or liquid.

76. The microalgal product of claim 75, which is dairy product-free, a LACT-ICE, an ICE-MILK, or an ICE-CREAM,
optionally wherein the microalgal product comprising one or more of an excipient, an antioxidant, an emulsifier, and a thickener, and/or one or more of fruit juice and flavoring, and
optionally wherein the microalgal product is in a sheet-like form.

77. A method for manufacturing an oil immersed product, comprising:
a step of preparing a microalgal concentrate by the method of claim 1; and
a step of mixing the microalgae with oil.

78. The method of claim 77, comprising a step of adding water to the microalgal concentrate and desalinating.

79. The method of claim 77, comprising a step of lyophilizing the microalgal concentrate.

80. The microalgal product of claim 66, which is an oil immersed product.

81. The oil immersed product of claim 80, comprising an antioxidant.

82. The oil immersed product of claim 81, wherein the antioxidant comprises α-tocopherol.

83. The oil immersed product of claim 80, comprising about 1 to 100% by weight of oil with respect to 1 g of dry algal body.

84. The oil immersed product of claim 80, comprising an emulsifier.

85. A capsule comprising the oil immersed product of claim 80.

86. The microalgal product of any one of claim 66, which is a dried product comprising one or more of a desiccant and an antioxidant.

87. A lightproof container enclosing the microalgal product of claim 66.
